(19) United States
(12) Patent Application Publication
(10) Pub. No.: US 2026/0193297 A1
(43) Pub. Date: Jul. 9, 2026

(54) NEW PEPTIDES AND USE THEREOF AS DELIVERY SYSTEMS FOR INTERNALIZATION OF MOLECULES OF INTEREST INTO TARGET CELLS

(71) Applicants: UNIVERSITE GRENOBLE ALPES, SAINT MARTIN D'HERES (FR); CENTRE NATIONAL DE LA RECHERCHE SCIENTIFIQUE, PARIS (FR); INSTITUT POLYTECHNIQUE DE GRENOBLE, GRENOBLE (FR)

(72) Inventors: Jean-Luc LENORMAND, Meylan (FR); Déborah REYNAUD, Voiron (FR)

(73) Assignees: UNIVERSITE GRENOBLE ALPES, SAINT MARTIN D'HERES (FR); CENTRE NATIONAL DE LA RECHERCHE SCIENTIFIQUE, Paris (FR); INSTITUT POLYTECHNIQUE DE GRENOBLE, Grenoble (FR)

(21) Appl. No.: 19/132,319

(22) PCT Filed: Dec. 8, 2023
(86) PCT No.: PCT/FR2023/051961

§ 371 Date: May 22, 2025

(30) Foreign Application Priority Data
Dec. 8, 2022 (FR) FR22/13003

(51) Int. Cl. C07K 14/00; A61K 38/00; A61K 47/64; A61K 49/00; A61P 35/00; C12N 15/62; C12N 15/63

(52) U.S. Cl. CPC C07K 14/00; A61K 38/00; A61K 47/64; A61K 49/0002; A61P 35/00; C12N 15/62; C12N 15/63; C07K 2319/00

(57) ABSTRACT

New cell-penetrating peptides including or consisting of an amino acid sequence selected from among the sequences SEQ ID NO: 2, SEQ ID NO: 3, SEQ ID NO: 4, SEQ ID NO: 5, SEQ ID NO: 6, SEQ ID NO: 7 and SEQ ID NO: 8. Further, fusion proteins including the peptides and a molecule of pharmaceutical, diagnostic or biotechnological interest.

### TECHNICAL FIELD OF THE INVENTION

The invention relates to the field of peptide delivery systems for internalizing molecules of interest in target cells. More particularly, it relates to novel peptides or nucleic acids encoding such peptides, and uses thereof as delivery systems for internalizing molecules of interest in target cells. It also relates to the combination of such peptides with molecules of interest, in particular polypeptides of interest and the use of such a combination in the treatment, diagnosis or prevention of pathologies, in particular cancer. The invention further relates to a fusion protein comprising a novel peptide having the cell-penetrating property (so-called “Cell-penetrating peptide, CPP”) object of the present invention and a polypeptide of interest, as well as a nucleic acid encoding such a fusion protein, an expression vector comprising nucleic acids encoding such a fusion protein, but also a host cell comprising such an expression vector. Finally, the invention also relates to a pharmaceutical composition comprising a novel peptide object of the present invention and a polypeptide of interest.

### PRIOR ART

Medicines derived from biotechnologies, also called biomedicines or biological medicines, have an important place in the treatment of human pathologies. They comprise in particular therapeutic proteins (enzymes, growth hormones, monoclonal antibodies, growth factors, recombinant vaccines, etc.), nucleic acids (DNA, siRNA, mRNA, oligonucleotides, etc.), peptides and derivatives such as peptide nucleic acids (PNA).

In some cases, it is necessary to use delivery systems for these biomedicines to be internalized in target cells. The internalization of therapeutic molecules into cells is the subject-matter of numerous researches aiming to find novel delivery systems, improving the effectiveness of the known delivery systems but also of the transported therapeutic molecules, improving targeting thereof at the cells and decreasing the effective dose of some treatments in order to reduce the risk of toxicity.

Several families of delivery peptides have already been discovered. These natural or synthetic peptides called PTD (standing for “Protein Transduction Domain”) or CPP (standing for “Cell-Penetrating Peptides”) have the ability to transport and transfer into the cells molecules such as peptides or nucleic acids according to different possible cell internalization mechanisms like, for example, endocytosis, macropinocytosis or membrane pore formation.

In their publication of 2008 “Expression and purification of Zebra Fusion Proteins and Applications for the Delivery of Macromolecules into Mammalian Cells” in the journal Current Protocols in Protein Science, 54:18.11.1-18.11.29, Lenormand and Rothe describe a method for producing fusion proteins comprising a segment of the ZEBRA protein and the eGFP protein or β-galactosidase. Said fusion proteins can be internalized in Hela cells at a concentration of 0.01 UM to 0.3 μM.

The ZEBRA protein is a transcriptional activator derived from the Epstein-Barr virus. It is a 245 amino acid protein comprising an N-terminal transactivation region (TAD), a DNA binding domain (DBD) and a leucine zipper type dimerization region (DIM). The C-terminal domain of said protein interacts with the zipper leucine domain leading to the formation of a hydrophobic pocket which stabilizes the ZEBRA/DNA protein complex.

Hitherto, the internalization routes taken by the delivery peptides, known to a person skilled in the art, such as endocytosis and macropinocytosis, have required a considerable energy expenditure in order to carry out this intracellular penetration mechanism. In addition, the endocytosis mechanism leads to the internalization of the molecules through endosomes whose contents undergo a partial degradation. Only a small fraction of the transported peptides is released into the cytosol, after break-up of the membrane of the endosomes, enabling them to exert their action at the intracellular level. Consequently, on an industrial production scale, in order to ensure the effectiveness of the transduction of polypeptides of interest, it is necessary to produce a large amount of delivery system and polypeptides of interest. This sometimes requires a heavy production or purification procedure that is not feasible for all types of polypeptides of interest. Moreover, for some polypeptides of interest, it is necessary to limit the administered amount because of the risks of toxicity. Hence, it seems to be necessary to use delivery polypeptides that are more effective in terms of cell-penetrating capacity in order to limit these risks.

The patent application WO2011135222 discloses the use of a peptide fragment derived from the ZEBRA protein (from the amino acid at position 170 to the amino acid at position 220) as a delivery peptide allowing performing an internalization of polypeptides of interest at low concentrations and avoiding the degradation of these polypeptides of interest by an endosomes-independent direct penetration mechanism.

The publication “MD11 mediated delivery of recombinant elF3f induces melanoma and colorectal carcinoma cell death” by R. MARCHIONE et al. in 2015 discloses the use of a complex formed by the elF3f protein fused with a cell-penetrating peptide to increase the available intracellular amount of elF3f protein in cancer cells and activate apoptosis thereof. The cell-penetrating peptide used is a sequence fragment of the ZEBRA protein (from the amino acid at position 178 to the amino acid at position 220) called MD11.

To date, there is still a need to find novel delivery peptides whose cell-penetrating potential is improved and the production of which is facilitated, these having to enable transport of the molecules of interest into the target cells at a low concentration, with a high efficiency and a better affinity, while ensuring good stability and low toxicity thereof in said cells.

### DISCLOSURE OF THE INVENTION

An objective of the present invention is to provide novel peptides as delivery systems for the internalization of molecules of interest in target cells. These new peptides are mutants of a peptide fragment of the ZEBRA protein. This fragment whose sequence is the sequence SEQ ID NO: 1 corresponding to the peptide sequence of the ZEBRA protein from the amino acid at position 178 to the amino acid at position 220 is known and will be referred to as MD11 in the remainder of the present description. This consists of a cell-penetrating peptide (CPP, standing for “cell-penetrating peptide”), also referred to as “delivery peptide”, or more simply “delivery system” or “delivery vector” (“Delivery system”).

To this end, according to a first aspect, the present invention relates to a peptide comprising or consisting of an amino acid sequence selected from among the sequences SEQ ID NO: 2, SEQ ID NO: 3, SEQ ID NO: 4, SEQ ID NO: 5, SEQ ID NO: 6, SEQ ID NO: 7 and SEQ ID NO: 8.

Such peptides may be qualified as delivery peptides and are intended for the internalization of molecules of interest in target cells. They have the advantage of benefiting from a cell-penetrating capacity at least equivalent to or higher than that of the MD11 transport peptide. Moreover, they are present in the cells even when they are treated with low concentrations in the nanomolar scale, which corroborates a direct and intermediate penetration into the cells for the molecules that they could carry. Finally, no toxicity of the peptides alone for the target cells could be highlighted.

By cell-penetrating capacity, it should be understood the efficiency of internalization of molecules of interest, i.e. the percentage of treated cells containing the molecules of interest in their cytoplasm. This internalization efficiency is based on the detection of molecules of interest in transduced cells, for example by means of microscopy or flow cytometry or any other imaging techniques allowing visualizing the internalization at the molecular level, for example bioluminescence resonance energy transfer (BRET, standing for “Bioluminescence Resonance Energy Transfer”), Forster-type resonance energy transfer (FRET, standing for “Förster Resonance Energy Transfer”), electron microscopy, atomic force microscopy (AFM, standing for “Atomic Force Microscopy”), optogenetics, etc.

The expression “delivery system” refers to a molecule capable of transporting and transferring another different molecule through the plasma membrane to enable it to penetrate the cell.

The expression “internalizing a molecule of interest in the target cells” refers to the passage of a molecule of interest from outside a target cell therein.

A second aspect of the present invention relates to a nucleic acid molecule encoding a peptide comprising or consisting of an amino acid sequence selected from among SEQ ID NO: 2, SEQ ID NO: 3, SEQ ID NO: 4, SEQ ID NO: 5, SEQ ID NO: 6, SEQ ID NO: 7 and SEQ ID NO: 8.

The nucleic acid sequences encoding the peptides represented by the sequences SEQ ID NO: 2, SEQ ID NO: 3, SEQ ID NO: 4, SEQ ID NO: 5, SEQ ID NO: 6, SEQ ID NO: 7 and SEQ ID NO: 8 as described hereinabove may be deduced from these amino acid sequences according to the principle of degeneration of the genetic code known to a person skilled in the art.

According to a third aspect, the present invention relates to an expression vector comprising a nucleic acid molecule encoding a peptide comprising or consisting of an amino acid sequence selected from among the sequences SEQ ID NO: 2, SEQ ID NO: 3, SEQ ID NO: 4, SEQ ID NO: 5, SEQ ID NO: 6, SEQ ID NO: 7 and SEQ ID NO: 8. Such an expression vector may be of any type known per se for implementation in genetic engineering, in particular a plasmid, a cosmid, a virus, a bacteriophage, containing the elements necessary for the transcription and translation of the sequence encoding the peptide according to the invention.

According to a particular embodiment of the present invention, the vector further includes the genetic engineering elements, in particular the replication origins and the promoters, allowing controlling the autonomous replication of the vector in the host organism and the specific expression of the peptides as described hereinabove.

A fourth aspect of the present invention relates to a host cell comprising a nucleic acid molecule encoding a peptide comprising or consisting of an amino acid sequence selected from among the sequences SEQ ID NO: 2, SEQ ID NO: 3, SEQ ID NO: 4, SEQ ID NO: 5, SEQ ID NO: 6, SEQ ID NO: 7 and SEQ ID NO: 8, or an expression vector comprising such a nucleic acid molecule. For example, the host cells man be prokaryotic cells such as E. coli or Bacillus, or eukaryotic cells such as yeasts in particular Saccharomyces cerevisiae and Pichia pastoris, filamentous fungi, including Trichoderma reesei and Aspergillus niger, insect cells using Baculoviruses, or cell lines such as CHO, HEK 293, Cos or Per.C6

According to a fifth aspect, the present invention relates to the use of a peptide comprising or consisting of an amino acid sequence selected from among the sequences SEQ ID NO: 2, SEQ ID NO: 3, SEQ ID NO: 4, SEQ ID NO: 5, SEQ ID NO: 6, SEQ ID NO: 7 and SEQ ID NO: 8 or a nucleic acid molecule encoding such a peptide or an expression vector comprising such a nucleic acid molecule or a host cell comprising such a nucleic acid molecule or such an expression vector, for obtaining a delivery system intended for internalizing a molecule of interest in target cells.

The cells that are likely to be the target cells of an internalization process implemented by a delivery system object of the present invention are preferably selected from among eukaryotic cells, in particular human cells. These human cells may be tumor cells, such as melanoma cells, breast cancer cells, glioblastoma cells, colon cancer cells, lymphomas cells. These human cells may also be normal cells including fibroblasts, epithelial cells, lymphocytes, dendritic cells, muscle cells like myocytes, myoblasts and myotubes for example, etc. These human cells may also be differentiated somatic cells which will be brought to dedifferentiation to form induced pluripotent stem cells (iPSCs). To target some cell lines, peptide sequences like homing peptides or target peptides, nuclear localization signals or NLS (standing for “nuclear localization signal”) may be grafted onto the delivery system according to the invention.

A sixth aspect of the present invention relates to a combination comprising a delivery system and a molecule of interest, said delivery system being a peptide comprising or consisting of an amino acid sequence selected from among the sequences SEQ ID NO: 2, SEQ ID NO: 3, SEQ ID NO: 4, SEQ ID NO: 5, SEQ ID NO: 6, SEQ ID NO: 7 and SEQ ID NO: 8.

In particular embodiments of the present invention, the molecule of interest is preferably a biotechnological, diagnosis or therapeutic molecule of interest. Preferably, such a molecule of interest is selected from among polypeptides, DNA, RNA, oligonucleotides, siRNA, shRNA miRNA, antisense RNA, or peptide nucleic acids (ANPs).

A seventh aspect of the present invention relates to a fusion protein comprising a delivery system which is a peptide comprising or consisting of an amino acid sequence selected from among SEQ ID NO: 2, SEQ ID NO: 3, SEQ ID NO: 4, SEQ ID NO: 5, SEQ ID NO: 6, SEQ ID NO: 7 and SEQ ID NO: 8, and a molecule of interest which is a polypeptide of interest.

By “fusion protein”, it should be understood a recombinant or synthetic polypeptide containing at least two peptides, derived from two different proteins, bonded together directly by a peptide bond, or by a peptide linker, for example GSGG. The polypeptide of interest may be bonded to the N-terminal or C-terminal portion of the delivery system.

According to a preferred embodiment of the present invention, the fusion protein comprises or consists of an amino acid sequence selected from among the sequences SEQ ID NO: 23 to SEQ ID NO: 29, SEQ ID NO: 31 to SEQ ID NO: 37, SEQ ID NO: 39 to SEQ ID NO: 45, SEQ ID NO: 47 to SEQ ID NO: 53 and SEQ ID NO: 55 to SEQ ID NO: 61.

According to an eighth aspect, the present invention relates to a nucleic acid molecule encoding the fusion protein object of the present invention.

According to a ninth aspect, the present invention relates to an expression vector comprising a nucleic acid molecule encoding the fusion protein object of the present invention. Such an expression vector may be of any type known per se for implementation in genetic engineering, in particular a plasmid, a cosmid, a virus, a bacteriophage, containing the elements necessary for the transcription and translation of the sequence encoding the fusion protein according to the invention.

According to a particular embodiment of the present invention, the vector further includes the genetic engineering elements, in particular the replication origins and the promoters, allowing controlling the autonomous replication of the vector in the host organism and the specific expression of the fusion proteins as described hereinabove.

According to a tenth aspect, the present invention relates to a host cell comprising a nucleic acid molecule encoding the fusion protein object of the present invention or an expression vector comprising such a nucleic acid molecule. For example, the host cells may be prokaryotic cells such as E. coli or Bacillus, or eukaryotic cells such as yeasts in particular Saccharomyces cerevisiae and Pichia pastoris, filamentous fungi, in particular Trichoderma reesei and Aspergillus niger, insect cells using Baculoviruses, or cell lines such as CHO, HEK 293, Cos, Per.C6

In particular embodiments of the combination or of the fusion protein object of the present invention, the molecule of interest is bonded to the delivery system by a covalent or non-covalent bond, such as an ionic bond, a hydrogen bond, or a hydrophobic bond. When the molecule of interest is a polypeptide of interest, according to an advantageous embodiment of the invention, the polypeptide of interest is bonded to the delivery system according to the invention by a direct peptide bond.

According to an eleventh aspect, the present invention relates to a pharmaceutical composition comprising a combination or a fusion protein object of the present invention. Preferably, the composition comprises a pharmaceutically-acceptable excipient and/or carrier. The selection of a pharmaceutically-acceptable excipient and/or carrier is known to a person skilled in the art.

According to a twelfth aspect, the present invention relates to the combination or the fusion protein or the pharmaceutical composition object of the present invention for use thereof in the treatment, diagnosis or prevention of cancers such as melanomas, breast cancer, brain tumors, glioblastomas, colon cancer, lymphomas.

For any of the aforementioned aspects of the invention, according to particular embodiments of the invention, the molecule of interest comprises or consists of a polypeptide of interest selected from among a polypeptide encoding the eGFP protein represented by the sequence SEQ ID NO: 17, a polypeptide encoding the elF3f protein represented by the sequence SEQ ID NO: 18, a polypeptide encoding the FERM protein represented by the sequence SEQ ID NO: 19, a polypeptide encoding all or part of the MDA-7 protein represented respectively by the sequences SEQ ID NO: 20 (whole MDA-7 protein) and SEQ ID NO: 21 (truncated MDA-7 protein) and a polypeptide represented by a sequence having 80%, in particular 90%, particularly 95% sequence identity with one of the sequences SEQ ID NO: 17, SEQ ID NO: 18, SEQ ID NO: 19, SEQ ID NO: 20 and SEQ ID NO: 21.

The percentage of identity of peptide sequences is determined by direct comparison of two sequences of polypeptide molecules, by determining the number of identical amino acid residues in the two sequences, and then by dividing it by the number of amino acid residues of the longest sequence of the two, and by multiplying the result by 100.

The nucleic acid sequences encoding the polypeptides represented by the sequences SEQ ID NO: 17, SEQ ID NO: 18, SEQ ID NO: 19, SEQ ID NO: 20, SEQ ID NO: 21 as described hereinabove may be deduced from these amino acid sequences of the peptides according to the principle of degeneration of the genetic code known to a person skilled in the art.

### BRIEF DESCRIPTION OF THE FIGURES

The invention will be better understood upon reading the following description, given as a non-limiting example, and made with reference to the figures which represent:

FIG. 1 illustrates a histogram showing the percentages of fluorescent HEK, B16-Ova and beta-TC-6 cells after treatment with fluorescent fusion proteins of the present invention;

FIG. 2 illustrates images of HEK and B16-Ova cells observed under a fluorescence microscope after treatment with fluorescent fusion proteins object of the present invention;

FIG. 3 illustrates viability curves of the B16-Ova and HEK cells as a function of the treatment dose after 24 h and 48 h allowing calculating the IC50 of the potentially therapeutic fusion proteins object of the present invention.

### DESCRIPTION OF THE EMBODIMENTS

In the remainder of the description, the nucleic acid sequences encoding the peptides and polypeptides represented by the amino acid sequences as described hereinafter, can be deduced from these amino acid sequences according to the principle of the degeneration of the genetic code known to a person skilled in the art.

### Making of the Plasmids MDmut and MD11

Starting from the original sequence of the cell-penetrating peptide (CPP) MD11, the Inventors have created 7 sequence modifications numbered from #2 to #8 according to the distancing from the original sequence:
SEQ ID NO: 2 called MDmut1DelCys which corresponds to the sequence KRYKNRVASRKRAKFKQLLQHYREVAAAKSSENDRLRLLLK,
SEQ ID NO: 3 called MDmut2 which corresponds to the sequence RREKNRVAARKCRAKFKNLLQHYREVAAAKSSENDRLRLLLK,
SEQ ID NO: 4 called MDmut2DelCys which corresponds to the sequence RREKNRVAARKRAKFKNLLQHYREVAAAKSSENDRLRLLLK,
SEQ ID NO: 5 called MDmut3 which corresponds to the sequence KRYKNRVASRKCRAKFKQAETQKLISEIDLLRKQNEQLKHKLEQL,
SEQ ID NO: 6 called MDmut3DelCys which corresponds to the sequence KRYKNRVASRKRAKFKQAETQKLISEIDLLRKQNEQLKHKLEQL,
SEQ ID NO: 7 called MDmut4 which corresponds to the sequence RREKNRVAARKCRAKFKNAETQKLISEIDLLRKQNEQLKHKLEQL,
SEQ ID NO: 8 called MDmut4DelCys which corresponds to the sequence RREKNRVAARKRAKFKNAETQKLISEIDLLRKQNEQLKHKLEQL.

The DNA fragments encoding each mutant CPP MDmut are obtained by PCR and inserted into the expression vector pET15b which allows expression in bacteria, the peptides whose N-terminal end is bound to a 6-histidine tag, all of the plasmids being constructed according to the same organization of genetic engineering elements surrounding the sequence of the CPP.

The sequence of MD11 numbered #1 and corresponding to the sequence KRYKNRVASRKCRAKFKQLLQHYREVAAAKSSENDRLRLLLKQ (SEQ ID NO: 1) is also inserted into an expression plasmid pET_15b comprising the same organization of genetic engineering elements as the plasmids comprising the MDmut mutants.

The organization of the genetic engineering elements is as follows in each expression plasmid in the direction 5′ to 3′:
Ndel Res Site-His Tag-TEV seq-MMP2 cleavage site-MD Seq-Xhol

### Res Site

wherein:
Nled Res Site=Ndel restriction site
His Tag=polyhistidine tag
TEV seq=cleavage sequence of the Tev protease
MMP2 cleavage site=metalloprotease 2 (tumor targeting) cleavage sequence
MD Seq=the sequence MD11 or MDmut
Xhol Res site=Xhol restriction site

All plasmids have been verified by two-way sequencing at the insertion sequence of the genetic elements encoding the fusion proteins of interest.

With this organization of the genetic engineering elements, the following sequences of cell-penetrating peptides (CPP) are thus obtained:
SEQ ID NO: 9 which corresponds to the sequence MHHHHHHHHENLYFQSGALGLPKRYKNRVASRKCRAKFKQLLQHYREVAAAKSSE NDRLRLLLKQ for the CPP comprising the sequence MD11,
SEQ ID NO: 10 which corresponds to the sequence MHHHHHHHHENLYFQSGALGLPKRYKNRVASRKRAKFKQLLQHYREVAAAKSSEN DRLRLLLK for the CPP comprising the sequence MDmut1DelCys,
SEQ ID NO: 11 which corresponds to the sequence MHHHHHHHHENLYFQSGALGLPRREKNRVAARKCRAKFKNLLQHYREVAAAKSSE NDRLRLLLK for the CPP comprising the sequence Mdmut2,
SEQ ID NO: 12 which corresponds to the sequence MHHHHHHHHENLYFQSGALGLPRREKNRVAARKRAKFKNLLQHYREVAAAKSSEN DRLRLLLK for the CPP comprising the sequence Mdmut2DelCys,
SEQ ID NO: 13 which corresponds to the sequence MHHHHHHHHENLYFQSGALGLPKRYKNRVASRKCRAKFKQAETQKLISEIDLLRKQ NEQLKHKLEQL for the CPP comprising the sequence Mdmut3,
SEQ ID NO: 14 which corresponds to the sequence MHHHHHHHHENLYFQSGALGLPKRYKNRVASRKRAKFKQAETQKLISEIDLLRKQN EQLKHKLEQL for the CPP comprising the sequence Mdmut3DelCys,
SEQ ID NO: 15 which corresponds to the sequence MHHHHHHHHENLYFQSGALGLPRREKNRVAARKCRAKFKNAETQKLISEIDLLRKQ NEQLKHKLEQL for the CPP comprising the sequence Mdmut4,
SEQ ID NO: 16 which corresponds to the sequence MHHHHHHHHENLYFQSGALGLPRREKNRVAARKRAKFKNAETQKLISEIDLLRKQN EQLKHKLEQL for the CPP comprising the sequence Mdmut4DelCys.

Making the plasmids of the polypeptides of interest

In order to obtain the sequences of the reporter molecules of interest, in this case polypeptides of the eGFP (standing for “enhanced green fluorescent protein”) proteins for biotechnological use and the elF3f (standing for “eukaryotic Translation Initiation Factor 3 subunit F”), FERM (corresponding to the FERM domain) proteins and MDA-7 proteins (standing for “Melanoma Differentiation Associated 7”) proteins, whole or truncated, also known under the name IL-24, for therapeutic use, to be fused with our Mdmut mutant CPPs, 5 plasmids, so-called donors, containing the inserts of interest wherein the sequences of the polypeptides of interest are as follows have been used:
SEQ ID NO: 17 corresponding to the polypeptide MVSKGEELFTGVVPILVELDGDVNGHKFSVSGEGEGDATYGKLTLKFICTTGKLPVP WPTLVTTLTYGVQCFSRYPDHMKQHDFFKSAMPEGYVQERTIFFKDDGNYKTRAE VKFEGDTLVNRIELKGIDFKEDGNILGHKLEYNYNSHNVYIMADKQKNGIKVNFKIRH NIEDGSVQLADHYQQNTPIGDGPVLLPDNHYLSTQSALSKDPNEKRDHMVLLEFVT AAGITLGMDELYK of the eGFP protein,
SEQ ID NO: 18 corresponding to the polypeptide ATPAVPVSAPPATPTPVPAAAPASVPAPTPAPAAAPVPAAAPASSSDPAAAAAATA APGQTPASAQAPAQTPAPALPGPALPGPFPGGRVVRLHPVILASIVDSYERRNEGA ARVIGTLLGTVDKHSVEVTNCFSVPHNESEDEVAVDMEFAKNMYELHKKVSPNELIL GWYATGHDITEHSVLIHEYYSREAPNPIHLTVDTSLQNGRMSIKAYVSTLMGVPGRT MGVMFTPLTVKYAYYDTERIGVDLIMKTCFSPNRVIGLSSDLQQVGGASARIQDALS TVLQYAEDVLSGKVSADNTVGRFLMSLVNQVPKIVPDDFETMLNSNINDLLMVTYLA NLTQSQIALNEKLVNL of the elF3f protein,
SEQ ID NO: 19 corresponding to the polypeptide MPKPINVRVTTMDAELEFAIQPNTTGKQLFDQVVKTIGLREVWYFGLHYVDNKGFPT WLKLDKKVSAQEVRKENPLQFKFRAKFYPEDVAEELIQDITQKLFFLQVKEGILSDEI YCPPETAVLLGSYAVQAKFGDYNKEVHKSGYLSSERLIPQRVMDQHKLTRDQWED RIQVWHAEHRGMLKDNAMLEYLKIAQDLEMYGINYFEIKNKKGTDLWLGVDALGLNI YEKDDKLTPKIGFPWSEIRNISFNDKKFVIKPIDKKAPDFVFYAPRLRINKRILQLCMG NHELYMRRRKPDTIEVQQMKAQAREEKHQKQLER of the FERM protein,
SEQ ID NO: 20 corresponding to the polypeptide NFQQRLQSLWTLARPFCPPLLATASQMQMVVLPCLGFTLLLWSQVSGAQGQEFHF GPCQVKGVVPQKLWEAFWAVKDTMQAQDNNTSCRLLQQEGLQNVSDAESCYLVH TLLEFYLKTVFKNYHNRTVEVRTLKSFSTLANNFVLIVSQLQPSQENEMFSIRDSAHR RFLLFRRAFKQLDVEAALTKALGEVDILLTWMQKFYKL of the MDA-7 protein,
SEQ ID NO: 21 corresponding to the polypeptide GQEFHFGPCQVKGVVPQKLWEAFWAVKDTMQAQDNITSARLLQQEVLQNVSDAE SCYLVHTLLEFYLKTVFKNHHNRTVEVRTLKSFSTLANNFVLIVSQLQPSQENEMFSI RDSAHRRFLLFRRAFKQLDVEAALTKALGEVDILLTWMQKFYKL of the truncated MDA-7 protein.

The Inventors have these sequences (SEQ ID NO: 17 to SEQ ID NO: 21) flanked by the Xhol and BamHI restriction sites in a pET15b plasmid. They have been sequenced beforehand thanks to the commercial primer pET_RP and their restriction profile has also been analyzed by simple and double digestions in order to ensure the possibility of extracting them without impacting the sequence that will have to be translated.

### Creation of the Plasmids Library

First, the 8 Mdmut plasmids and the 5 donor plasmids of the polypeptides of interest have been transformed into E. coli XL10 Gold bacteria. For this purpose, 25 μl of bacteria have been transformed with 50 ng of plasmids by heat shock 30 min in ice and then 40 sec at 42° C. before passage through the ice. This step has been followed by a liquid culture in 100 μL of SOC culture medium at 37° C. for 1 h and then spreading on a Petri dish containing LB (Lysogeny Broth)-Agar-Ampicillin medium and incubation at 37° C. overnight. The next day, an isolated colony has been seeded in 5 ml of LB-Ampicillin medium and incubated at 37° C.-150 rpm overnight. Afterwards, 2 ml of culture have been centrifuged at 11,000 g and the plasmids have been purified and eluted in 25 μL of water using the Miniprep kit from Macherey Nagel according to the protocol of the manufacturer.

The plasmids thus obtained have been prepared for subcloning after double digestion by the restriction enzymes Xhol and BamHI 2 μg plasmid have therefore been digested with 5 units of each restriction enzyme for 3 h at 37° C. The migration of the digestion products in a 0.6% agarose gel-TAE 0.5×-Gel Red 1× for 1 h30 at 75V has been followed by a purification of the bands of interest using the PCR and Gel Clean kit from Macherey Nagel according to the protocol of the manufacturer.

The MDmut and MD11 plasmids, which could be qualified as recipients, have given their backbone containing the CPP-mutants or MD11 sequences and the plasmids comprising the polypeptides of interest, qualified as donors, have given their insert containing the sequence of the polypeptides of interest.

It has been possible to subclone the inserts corresponding to the polypeptides of interest into each of the 8 MDmut plasmids, thereby creating a new library of 40 recombinant plasmids enabling the synthesis of the fusion proteins. For this purpose, a ligation has been performed with 3 molar fold of each insert of polypeptides of interest and 50 ng of mutant plasmid thanks to a T4 DNA ligase in 10 min at 22° C. This step has been followed by the transformation of 25 μL of XL10 Gold bacteria with half of the ligation product according to the same protocol as mentioned before.

The colonies transformed by the recombinant plasmids have been pre-selected thanks to colony PCRs. To do so, a ready-to-use 2× commercial enzyme has been used by half diluting therein 10 μM of forward and reverse primers in sterile water. A cone tap of all colonies of the dish has been performed in order to contaminate 20 μl of PCR mix before launching the thermocycler according to a program adapted to the primers (Lid=110° C.; 1: 3 min—94°; 2:30 sec—94°; 3:30 sec—60° C.; 4:1 min—72° C.; 5: GOTO 2×25 repeats; 6: 5 min—72° C.; 7: Hold-16° C.). Next, the migration of the PCR products into an agarose gel 1.5%-TAE 0.5×—Gel Red 1× for 40 min at 100V has been performed.

After migration of the PCR products onto the agarose gel, the samples derived from the colony taps have been compared to the positive control samples (donor plasmid) or negative control (Water). Afterwards, one or two colon (y/ies) having given an amplicon of size and appearance comparable to the positive control, have been cultured in order to amplify and purify, through the use of a commercial “miniprep” kit, the plasmids to send them to the two-way sequencing (T7 and pET_RP primers) for verification of the alignment of their sequence with respect to the theoretical sequence.

Afterwards, new E. coli XL10 Gold bacteria have been retransformed from the validated plasmid samples in sequencing. This new transformation allowed the constitution of a glycerol stock (freezing at −80° C. of 750 μl of overnight culture of XL10 bacteria with 25% sterile glycerol) and a stock of concentrated recombinant plasmids, and purified by the use of the “midiprep” Macherey Nagel kit according to the protocol of the manufacturer.

In each recombinant plasmid, the organization of the genetic engineering elements is as follows in the direction 5′ to 3′:
Ndel Res Site-His Tag-TEV seq-MMP2 cleavage site-MD Seq-Xhol-PolyPep-BamHI
wherein:
Nled Res site=Ndel restriction site
His Tag=polyhistidine tag
TEV seq=cleavage sequence of the Tev protease
MMP2 cleavage site=metalloprotease 2 (tumor targeting) cleavage sequence
MD Seq=the sequence MD11 (SEQ ID NO: 1) or a sequence MDmut (SEQ ID NO: 2 to SEQ ID NO: 8)
Xhol=Xhol restriction site
PolyPep=the sequence of the polypeptide of interest (SEQ ID NO: 17 to SEQ ID NO: 21)
BamHI=BamHI restriction site

Thus, the following sequences of fusion proteins encoded by the recombinant plasmids thus created are obtained:
SEQ ID NO: 22 which corresponds to the sequence MHHHHHHHHENLYFQSGALGLPKRYKNRVASRKCRAKFKQLLQHYREVAAAKSSE NDRLRLLLKQLEMVSKGEELFTGVVPILVELDGDVNGHKFSVSGEGEGDATYGKLT LKFICTTGKLPVPWPTLVTTLTYGVQCFSRYPDHMKQHDFFKSAMPEGYVQERTIFF KDDGNYKTRAEVKFEGDTLVNRIELKGIDFKEDGNILGHKLEYNYNSHNVYIMADKQ KNGIKVNFKIRHNIEDGSVQLADHYQQNTPIGDGPVLLPDNHYLSTQSALSKDPNEK RDHMVLLEFVTAAGITLGMDELYK for the CPP (SEQ ID NO: 9) comprising the genetic engineering elements and the sequence MD11, in fusion with the eGFP protein (SEQ ID NO: 17).
SEQ ID NO: 23 which corresponds to the sequence MHHHHHHHHENLYFQSGALGLPKRYKNRVASRKRAKFKQLLQHYREVAAAKSSEN DRLRLLLKLEMVSKGEELFTGVVPILVELDGDVNGHKFSVSGEGEGDATYGKLTLKF ICTTGKLPVPWPTLVTTLTYGVQCFSRYPDHMKQHDFFKSAMPEGYVQERTIFFKD DGNYKTRAEVKFEGDTLVNRIELKGIDFKEDGNILGHKLEYNYNSHNVYIMADKQKN GIKVNFKIRHNIEDGSVQLADHYQQNTPIGDGPVLLPDNHYLSTQSALSKDPNEKRD HMVLLEFVTAAGITLGMDELYK for the CPP (SEQ ID NO: 10) comprising the genetic engineering elements and the sequence MDmut1DelCys in fusion with the eGFP protein (SEQ ID NO: 17).
SEQ corresponds to the sequence ID NO: 24 which MHHHHHHHHENLYFQSGALGLPRREKNRVAARKCRAKFKNLLQHYREVAAAKSSE NDRLRLLLKLEMVSKGEELFTGVVPILVELDGDVNGHKFSVSGEGEGDATYGKLTLK FICTTGKLPVPWPTLVTTLTYGVQCFSRYPDHMKQHDFFKSAMPEGYVQERTIFFK DDGNYKTRAEVKFEGDTLVNRIELKGIDFKEDGNILGHKLEYNYNSHNVYIMADKQK NGIKVNFKIRHNIEDGSVQLADHYQQNTPIGDGPVLLPDNHYLSTQSALSKDPNEKR DHMVLLEFVTAAGITLGMDELYK for the CPP (SEQ ID NO: 11) comprising the genetic engineering elements and the sequence MDmut2 in fusion with the eGFP protein (SEQ ID NO: 17).
SEQ ID NO: 25 which corresponds to the sequence MHHHHHHHHENLYFQSGALGLPRREKNRVAARKRAKFKNLLQHYREVAAAKSSEN DRLRLLLKLEMVSKGEELFTGVVPILVELDGDVNGHKFSVSGEGEGDATYGKLTLKF ICTTGKLPVPWPTLVTTLTYGVQCFSRYPDHMKQHDFFKSAMPEGYVQERTIFFKD DGNYKTRAEVKFEGDTLVNRIELKGIDFKEDGNILGHKLEYNYNSHNVYIMADKQKN GIKVNFKIRHNIEDGSVQLADHYQQNTPIGDGPVLLPDNHYLSTQSALSKDPNEKRD HMVLLEFVTAAGITLGMDELYK for the CPP (SEQ ID NO: 12) comprising the genetic engineering elements and the sequence MDmut2DelCys in fusion with the eGFP protein (SEQ ID NO: 17).
SEQ ID NO: 26 which corresponds to the sequence MHHHHHHHHENLYFQSGALGLPKRYKNRVASRKCRAKFKQAETQKLISEIDLLRKQ NEQLKHKLEQLLEMVSKGEELFTGVVPILVELDGDVNGHKFSVSGEGEGDATYGKL TLKFICTTGKLPVPWPTLVTTLTYGVQCFSRYPDHMKQHDFFKSAMPEGYVQERTIF FKDDGNYKTRAEVKFEGDTLVNRIELKGIDFKEDGNILGHKLEYNYNSHNVYIMADK QKNGIKVNFKIRHNIEDGSVQLADHYQQNTPIGDGPVLLPDNHYLSTQSALSKDPNE KRDHMVLLEFVTAAGITLGMDELYK for the CPP (SEQ ID NO: 13) comprising the genetic engineering elements and the sequence MDmut3 in fusion with the eGFP protein (SEQ ID NO: 17).
SEQ ID NO: 27 which corresponds to the sequence MHHHHHHHHENLYFQSGALGLPKRYKNRVASRKRAKFKQAETQKLISEIDLLRKQN EQLKHKLEQLLEMVSKGEELFTGVVPILVELDGDVNGHKFSVSGEGEGDATYGKLT LKFICTTGKLPVPWPTLVTTLTYGVQCFSRYPDHMKQHDFFKSAMPEGYVQERTIFF KDDGNYKTRAEVKFEGDTLVNRIELKGIDFKEDGNILGHKLEYNYNSHNVYIMADKQ KNGIKVNFKIRHNIEDGSVQLADHYQQNTPIGDGPVLLPDNHYLSTQSALSKDPNEK RDHMVLLEFVTAAGITLGMDELYK for the CPP (SEQ ID NO: 14) comprising the genetic engineering elements and the sequence MDmut3DelCys in fusion with the eGFP protein (SEQ ID NO: 17).
SEQ ID NO: 28 which corresponds to the sequence MHHHHHHHHENLYFQSGALGLPRREKNRVAARKCRAKFKNAETQKLISEIDLLRKQ NEQLKHKLEQLLEMVSKGEELFTGVVPILVELDGDVNGHKFSVSGEGEGDATYGKL TLKFICTTGKLPVPWPTLVTTLTYGVQCFSRYPDHMKQHDFFKSAMPEGYVQERTIF FKDDGNYKTRAEVKFEGDTLVNRIELKGIDFKEDGNILGHKLEYNYNSHNVYIMADK QKNGIKVNFKIRHNIEDGSVQLADHYQQNTPIGDGPVLLPDNHYLSTQSALSKDPNE KRDHMVLLEFVTAAGITLGMDELYK for the CPP (SEQ ID NO: 15) comprising the genetic engineering elements and the sequence MDmut4 in fusion with the eGFP protein (SEQ ID NO: 17).
SEQ ID NO: 29 which corresponds to the sequence MHHHHHHHHENLYFQSGALGLPRREKNRVAARKRAKFKNAETQKLISEIDLLRKQN EQLKHKLEQLLEMVSKGEELFTGVVPILVELDGDVNGHKFSVSGEGEGDATYGKLT LKFICTTGKLPVPWPTLVTTLTYGVQCFSRYPDHMKQHDFFKSAMPEGYVQERTIFF KDDGNYKTRAEVKFEGDTLVNRIELKGIDFKEDGNILGHKLEYNYNSHNVYIMADKQ KNGIKVNFKIRHNIEDGSVQLADHYQQNTPIGDGPVLLPDNHYLSTQSALSKDPNEK RDHMVLLEFVTAAGITLGMDELYK for the CPP (SEQ ID NO: 16) comprising the genetic engineering elements and the sequence MDmut4DelCys in fusion with the eGFP protein (SEQ ID NO: 17).
SEQ ID NO: 30 which corresponds to the sequence MHHHHHHHHENLYFQSGALGLPKRYKNRVASRKCRAKFKQLLQHYREVAAAKSSE NDRLRLLLKQLEATPAVPVSAPPATPTPVPAAAPASVPAPTPAPAAAPVPAAAPASS SDPAAAAAATAAPGQTPASAQAPAQTPAPALPGPALPGPFPGGRVVRLHPVILASIV DSYERRNEGAARVIGTLLGTVDKHSVEVTNCFSVPHNESEDEVAVDMEFAKNMYEL HKKVSPNELILGWYATGHDITEHSVLIHEYYSREAPNPIHLTVDTSLQNGRMSIKAYV STLMGVPGRTMGVMFTPLTVKYAYYDTERIGVDLIMKTCFSPNRVIGLSSDLQQVG GASARIQDALSTVLQYAEDVLSGKVSADNTVGRFLMSLVNQVPKIVPDDFETMLNS NINDLLMVTYLANLTQSQIALNEKLVNL for the CPP (SEQ ID NO: 9) comprising the genetic engineering elements and the sequence MD11 in fusion with the protein elF3f (SEQ ID NO: 18).
SEQ ID NO: 31 which corresponds to the sequence MHHHHHHHHENLYFQSGALGLPKRYKNRVASRKRAKFKQLLQHYREVAAAKSSEN DRLRLLLKLEATPAVPVSAPPATPTPVPAAAPASVPAPTPAPAAAPVPAAAPASSSD PAAAAAATAAPGQTPASAQAPAQTPAPALPGPALPGPFPGGRVVRLHPVILASIVDS YERRNEGAARVIGTLLGTVDKHSVEVTNCFSVPHNESEDEVAVDMEFAKNMYELHK KVSPNELILGWYATGHDITEHSVLIHEYYSREAPNPIHLTVDTSLQNGRMSIKAYVST LMGVPGRTMGVMFTPLTVKYAYYDTERIGVDLIMKTCFSPNRVIGLSSDLQQVGGA SARIQDALSTVLQYAEDVLSGKVSADNTVGRFLMSLVNQVPKIVPDDFETMLNSNIN DLLMVTYLANLTQSQIALNEKLVNL for the CPP (SEQ ID NO: 10) comprising the genetic engineering elements and the sequence MDmut1DelCys in fusion with the protein elF3f (SEQ ID NO: 18).
SEQ ID NO: 32 which corresponds to the sequence MHHHHHHHHENLYFQSGALGLPRREKNRVAARKCRAKFKNLLQHYREVAAAKSSE NDRLRLLLKLEATPAVPVSAPPATPTPVPAAAPASVPAPTPAPAAAPVPAAAPASSS DPAAAAAATAAPGQTPASAQAPAQTPAPALPGPALPGPFPGGRVVRLHPVILASIVD SYERRNEGAARVIGTLLGTVDKHSVEVTNCFSVPHNESEDEVAVDMEFAKNMYELH KKVSPNELILGWYATGHDITEHSVLIHEYYSREAPNPIHLTVDTSLQNGRMSIKAYVS TLMGVPGRTMGVMFTPLTVKYAYYDTERIGVDLIMKTCFSPNRVIGLSSDLQQVGG ASARIQDALSTVLQYAEDVLSGKVSADNTVGRFLMSLVNQVPKIVPDDFETMLNSNI NDLLMVTYLANLTQSQIALNEKLVNL for the CPP (SEQ ID NO: 11) comprising the genetic engineering elements and the sequence MDmut2 in fusion with the protein elF3f (SEQ ID NO: 18).
SEQ ID NO: 33 which corresponds to the sequence MHHHHHHHHENLYFQSGALGLPRREKNRVAARKRAKFKNLLQHYREVAAAKSSEN DRLRLLLKLEATPAVPVSAPPATPTPVPAAAPASVPAPTPAPAAAPVPAAAPASSSD PAAAAAATAAPGQTPASAQAPAQTPAPALPGPALPGPFPGGRVVRLHPVILASIVDS YERRNEGAARVIGTLLGTVDKHSVEVTNCFSVPHNESEDEVAVDMEFAKNMYELHK KVSPNELILGWYATGHDITEHSVLIHEYYSREAPNPIHLTVDTSLQNGRMSIKAYVST LMGVPGRTMGVMFTPLTVKYAYYDTERIGVDLIMKTCFSPNRVIGLSSDLQQVGGA SARIQDALSTVLQYAEDVLSGKVSADNTVGRFLMSLVNQVPKIVPDDFETMLNSNIN DLLMVTYLANLTQSQIALNEKLVNL for the CPP (SEQ ID NO: 12) comprising the genetic engineering elements and the sequence MDmut2DelCys in fusion with the protein elF3f (SEQ ID NO: 18).
SEQ ID NO: 34 which corresponds to the sequence MHHHHHHHHENLYFQSGALGLPKRYKNRVASRKCRAKFKQAETQKLISEIDLLRKQ NEQLKHKLEQLLEATPAVPVSAPPATPTPVPAAAPASVPAPTPAPAAAPVPAAAPAS SSDPAAAAAATAAPGQTPASAQAPAQTPAPALPGPALPGPFPGGRVVRLHPVILASI VDSYERRNEGAARVIGTLLGTVDKHSVEVTNCFSVPHNESEDEVAVDMEFAKNMY ELHKKVSPNELILGWYATGHDITEHSVLIHEYYSREAPNPIHLTVDTSLONGRMSIKA YVSTLMGVPGRTMGVMFTPLTVKYAYYDTERIGVDLIMKTCFSPNRVIGLSSDLQQV GGASARIQDALSTVLQYAEDVLSGKVSADNTVGRFLMSLVNQVPKIVPDDFETMLN SNINDLLMVTYLANLTQSQIALNEKLVNL for the CPP (SEQ ID NO: 13) comprising the genetic engineering elements and the sequence MDmut3 in fusion with the protein elF3f (SEQ ID NO: 18).
SEQ ID NO: 35 which corresponds to the sequence MHHHHHHHHENLYFQSGALGLPKRYKNRVASRKRAKFKQAETQKLISEIDLLRKQN EQLKHKLEQLLEATPAVPVSAPPATPTPVPAAAPASVPAPTPAPAAAPVPAAAPASS SDPAAAAAATAAPGQTPASAQAPAQTPAPALPGPALPGPFPGGRVVRLHPVILASIV DSYERRNEGAARVIGTLLGTVDKHSVEVTNCFSVPHNESEDEVAVDMEFAKNMYEL HKKVSPNELILGWYATGHDITEHSVLIHEYYSREAPNPIHLTVDTSLQNGRMSIKAYV STLMGVPGRTMGVMFTPLTVKYAYYDTERIGVDLIMKTCFSPNRVIGLSSDLQQVG GASARIQDALSTVLQYAEDVLSGKVSADNTVGRFLMSLVNQVPKIVPDDFETMLNS NINDLLMVTYLANLTQSQIALNEKLVNL for the CPP (SEQ ID NO: 14) comprising the genetic engineering elements and the sequence MDmut3DelCys in fusion with the protein elF3f (SEQ ID NO: 18).
SEQ ID NO: 36 which corresponds to the sequence MHHHHHHHHENLYFQSGALGLPRREKNRVAARKCRAKFKNAETQKLISEIDLLRKQ NEQLKHKLEQLLEATPAVPVSAPPATPTPVPAAAPASVPAPTPAPAAAPVPAAAPAS SSDPAAAAAATAAPGQTPASAQAPAQTPAPALPGPALPGPFPGGRVVRLHPVILASI VDSYERRNEGAARVIGTLLGTVDKHSVEVTNCFSVPHNESEDEVAVDMEFAKNMY ELHKKVSPNELILGWYATGHDITEHSVLIHEYYSREAPNPIHLTVDTSLQNGRMSIKA YVSTLMGVPGRTMGVMFTPLTVKYAYYDTERIGVDLIMKTCFSPNRVIGLSSDLQQV GGASARIQDALSTVLQYAEDVLSGKVSADNTVGRFLMSLVNQVPKIVPDDFETMLN SNINDLLMVTYLANLTQSQIALNEKLVNL for the CPP (SEQ ID NO: 15) comprising the genetic engineering elements and the sequence MDmut4 in fusion with the protein elF3f (SEQ ID NO: 18).
SEQ ID NO: 37 which corresponds to the sequence MHHHHHHHHENLYFQSGALGLPRREKNRVAARKRAKFKNAETQKLISEIDLLRKQN EQLKHKLEQLLEATPAVPVSAPPATPTPVPAAAPASVPAPTPAPAAAPVPAAAPASS SDPAAAAAATAAPGQTPASAQAPAQTPAPALPGPALPGPFPGGRVVRLHPVILASIV DSYERRNEGAARVIGTLLGTVDKHSVEVTNCFSVPHNESEDEVAVDMEFAKNMYEL HKKVSPNELILGWYATGHDITEHSVLIHEYYSREAPNPIHLTVDTSLQNGRMSIKAYV STLMGVPGRTMGVMFTPLTVKYAYYDTERIGVDLIMKTCFSPNRVIGLSSDLQQVG GASARIQDALSTVLQYAEDVLSGKVSADNTVGRFLMSLVNQVPKIVPDDFETMLNS NINDLLMVTYLANLTQSQIALNEKLVNL for the CPP (SEQ ID NO: 16) comprising the genetic engineering elements and the sequence MDmut4DelCys in fusion with the protein elF3f (SEQ ID NO: 18).
SEQ ID NO: 38 which corresponds to the sequence MHHHHHHHHENLYFQSGALGLPKRYKNRVASRKCRAKFKQLLQHYREVAAAKSSE NDRLRLLLKQLEPKPINVRVTTMDAELEFAIQPNTTGKQLFDQVVKTIGLREVWYFG LHYVDNKGFPTWLKLDKKVSAQEVRKENPLQFKFRAKFYPEDVAEELIQDITQKLFF LQVKEGILSDEIYCPPETAVLLGSYAVQAKFGDYNKEVHKSGYLSSERLIPQRVMDQ HKLTRDQWEDRIQVWHAEHRGMLKDNAMLEYLKIAQDLEMYGINYFEIKNKKGTDL WLGVDALGLNIYEKDDKLTPKIGFPWSEIRNISFNDKKFVIKPIDKKAPDFVFYAPRLR INKRILQLCMGNHELYMRRRKPDTIEVQQMKAQAREEKHQKQLER for the CPP (SEQ ID NO: 9) comprising the genetic engineering elements and the sequence MD11 in fusion with the protein FERM (SEQ ID NO: 19).
SEQ ID NO: 39 which corresponds to the sequence MHHHHHHHHENLYFQSGALGLPKRYKNRVASRKRAKFKQLLQHYREVAAAKSSEN DRLRLLLKLEPKPINVRVTTMDAELEFAIQPNTTGKQLFDQVVKTIGLREVWYFGLHY VDNKGFPTWLKLDKKVSAQEVRKENPLQFKFRAKFYPEDVAEELIQDITQKLFFLQV KEGILSDEIYCPPETAVLLGSYAVQAKFGDYNKEVHKSGYLSSERLIPQRVMDQHKL TRDQWEDRIQVWHAEHRGMLKDNAMLEYLKIAQDLEMYGINYFEIKNKKGTDLWLG VDALGLNIYEKDDKLTPKIGFPWSEIRNISFNDKKFVIKPIDKKAPDFVFYAPRLRINKR ILQLCMGNHELYMRRRKPDTIEVQQMKAQAREEKHQKQLER for the CPP (SEQ ID NO: 10) comprising the genetic engineering elements and the sequence MDmut1DelCys in fusion with the protein FERM (SEQ ID NO: 19).
SEQ ID NO: 40 which corresponds to the sequence MHHHHHHHHENLYFQSGALGLPRREKNRVAARKCRAKFKNLLQHYREVAAAKSSE NDRLRLLLKLEPKPINVRVTTMDAELEFAIQPNTTGKQLFDQVVKTIGLREVWYFGLH YVDNKGFPTWLKLDKKVSAQEVRKENPLQFKFRAKFYPEDVAEELIQDITQKLFFLQ VKEGILSDEIYCPPETAVLLGSYAVQAKFGDYNKEVHKSGYLSSERLIPQRVMDQHK LTRDQWEDRIQVWHAEHRGMLKDNAMLEYLKIAQDLEMYGINYFEIKNKKGTDLWL GVDALGLNIYEKDDKLTPKIGFPWSEIRNISFNDKKFVIKPIDKKAPDFVFYAPRLRIN KRILQLCMGNHELYMRRRKPDTIEVQQMKAQAREEKHQKQLER for the CPP (SEQ ID NO: 11) comprising the genetic engineering elements and the sequence MDmut2 in fusion with the protein FERM (SEQ ID NO: 19).
SEQ ID NO: 41 which corresponds to the sequence MHHHHHHHHENLYFQSGALGLPRREKNRVAARKRAKFKNLLQHYREVAAAKSSEN DRLRLLLKLEPKPINVRVTTMDAELEFAIQPNTTGKQLFDQVVKTIGLREVWYFGLHY VDNKGFPTWLKLDKKVSAQEVRKENPLQFKFRAKFYPEDVAEELIQDITQKLFFLQV KEGILSDEIYCPPETAVLLGSYAVQAKFGDYNKEVHKSGYLSSERLIPQRVMDQHKL TRDQWEDRIQVWHAEHRGMLKDNAMLEYLKIAQDLEMYGINYFEIKNKKGTDLWLG VDALGLNIYEKDDKLTPKIGFPWSEIRNISFNDKKFVIKPIDKKAPDFVFYAPRLRINKR ILQLCMGNHELYMRRRKPDTIEVQQMKAQAREEKHQKQLER for the CPP (SEQ ID NO: 12) comprising the genetic engineering elements and the sequence MDmut2DelCys in fusion with the protein FERM (SEQ ID NO: 19).
SEQ ID NO: 42 which corresponds to the sequence MHHHHHHHHENLYFQSGALGLPKRYKNRVASRKCRAKFKQAETQKLISEIDLLRKQ NEQLKHKLEQLLEPKPINVRVTTMDAELEFAIQPNTTGKQLFDQVVKTIGLREVWYF GLHYVDNKGFPTWLKLDKKVSAQEVRKENPLQFKFRAKFYPEDVAEELIQDITQKLF FLQVKEGILSDEIYCPPETAVLLGSYAVQAKFGDYNKEVHKSGYLSSERLIPQRVMD QHKLTRDQWEDRIQVWHAEHRGMLKDNAMLEYLKIAQDLEMYGINYFEIKNKKGTD LWLGVDALGLNIYEKDDKLTPKIGFPWSEIRNISFNDKKFVIKPIDKKAPDFVFYAPRL RINKRILQLCMGNHELYMRRRKPDTIEVQQMKAQAREEKHQKQLER for the CPP (SEQ ID NO: 13) comprising the genetic engineering elements and the sequence MDmut3 in fusion with the protein FERM (SEQ ID NO: 19).
SEQ ID NO: 43 which corresponds to the sequence MHHHHHHHHENLYFQSGALGLPKRYKNRVASRKRAKFKQAETQKLISEIDLLRKQN EQLKHKLEQLLEPKPINVRVTTMDAELEFAIQPNTTGKQLFDQVVKTIGLREVWYFG LHYVDNKGFPTWLKLDKKVSAQEVRKENPLQFKFRAKFYPEDVAEELIQDITQKLFF LQVKEGILSDEIYCPPETAVLLGSYAVQAKFGDYNKEVHKSGYLSSERLIPQRVMDQ HKLTRDQWEDRIQVWHAEHRGMLKDNAMLEYLKIAQDLEMYGINYFEIKNKKGTDL WLGVDALGLNIYEKDDKLTPKIGFPWSEIRNISFNDKKFVIKPIDKKAPDFVFYAPRLR INKRILQLCMGNHELYMRRRKPDTIEVQQMKAQAREEKHQKQLER for the CPP (SEQ ID NO: 14) comprising the genetic engineering elements and the sequence MDmut3DelCys in fusion with the protein FERM (SEQ ID NO: 19).
SEQ ID NO: 44 which corresponds to the sequence ID NO: MHHHHHHHHENLYFQSGALGLPRREKNRVAARKCRAKFKNAETQKLISEIDLLRKQ NEQLKHKLEQLLEPKPINVRVTTMDAELEFAIQPNTTGKQLFDQVVKTIGLREVWYF GLHYVDNKGFPTWLKLDKKVSAQEVRKENPLQFKFRAKFYPEDVAEELIQDITQKLF FLQVKEGILSDEIYCPPETAVLLGSYAVQAKFGDYNKEVHKSGYLSSERLIPQRVMD QHKLTRDQWEDRIQVWHAEHRGMLKDNAMLEYLKIAQDLEMYGINYFEIKNKKGTD LWLGVDALGLNIYEKDDKLTPKIGFPWSEIRNISFNDKKFVIKPIDKKAPDFVFYAPRL RINKRILQLCMGNHELYMRRRKPDTIEVQQMKAQAREEKHQKQLER for the CPP (SEQ ID NO:15) comprising the genetic engineering elements and the sequence MDmut4 in fusion with the protein FERM (SEQ ID NO: 19).
SEQ ID NO: 45 which corresponds to the sequence MMHHHHHHHHENLYFQSGALGLPRREKNRVAARKRAKFKNAETQKLISEIDLLRKQ NEQLKHKLEQLLEPKPINVRVTTMDAELEFAIQPNTTGKQLFDQVVKTIGLREVWYF GLHYVDNKGFPTWLKLDKKVSAQEVRKENPLQFKFRAKFYPEDVAEELIQDITQKLF FLQVKEGILSDEIYCPPETAVLLGSYAVQAKFGDYNKEVHKSGYLSSERLIPQRVMD QHKLTRDQWEDRIQVWHAEHRGMLKDNAMLEYLKIAQDLEMYGINYFEIKNKKGTD LWLGVDALGLNIYEKDDKLTPKIGFPWSEIRNISFNDKKFVIKPIDKKAPDFVFYAPRL RINKRILQLCMGNHELYMRRRKPDTIEVQQMKAQAREEKHQKQLER for the CPP (SEQ ID NO: 16) comprising the genetic engineering elements and the sequence MDmut4DelCys in fusion with the protein FERM (SEQ ID NO: 19).
SEQ ID NO: 46 which corresponds to the sequence MHHHHHHHHENLYFQSGALGLPKRYKNRVASRKCRAKFKQLLQHYREVAAAKSSE NDRLRLLLKQLENFQQRLQSLWTLARPFCPPLLATASQMQMVVLPCLGFTLLLWSQ VSGAQGQEFHFGPCQVKGVVPQKLWEAFWAVKDTMQAQDNNTSCRLLQQEGLQ NVSDAESCYLVHTLLEFYLKTVFKNYHNRTVEVRTLKSFSTLANNFVLIVSQLQPSQ ENEMFSIRDSAHRRFLLFRRAFKQLDVEAALTKALGEVDILLTWMQKFYKL for the CPP (SEQ ID NO: 9) comprising the genetic engineering elements and the sequence MD11 in fusion with the protein MDA-7 (SEQ ID NO: 20).
SEQ ID NO: 47 which corresponds to the sequence MHHHHHHHHENLYFQSGALGLPKRYKNRVASRKRAKFKQLLQHYREVAAAKSSEN DRLRLLLKLENFQQRLQSLWTLARPFCPPLLATASQMQMVVLPCLGFTLLLWSQVS GAQGQEFHFGPCQVKGVVPQKLWEAFWAVKDTMQAQDNNTSCRLLQQEGLQNV SDAESCYLVHTLLEFYLKTVFKNYHNRTVEVRTLKSFSTLANNFVLIVSQLQPSQEN EMFSIRDSAHRRFLLFRRAFKQLDVEAALTKALGEVDILLTWMQKFYKL for the CPP (SEQ ID NO: 10) comprising the genetic engineering elements and the sequence MDmut1DelCys in fusion with the protein MDA-7 (SEQ ID NO: 20).
SEQ ID NO: 48 which corresponds to the sequence ID NO: MHHHHHHHHENLYFQSGALGLPRREKNRVAARKCRAKFKNLLQHYREVAAAKSSE NDRLRLLLKLENFQQRLQSLWTLARPFCPPLLATASQMQMVVLPCLGFTLLLWSQV SGAQGQEFHFGPCQVKGVVPQKLWEAFWAVKDTMQAQDNNTSCRLLQQEGLQN VSDAESCYLVHTLLEFYLKTVFKNYHNRTVEVRTLKSFSTLANNFVLIVSQLQPSQE NEMFSIRDSAHRRFLLFRRAFKQLDVEAALTKALGEVDILLTWMQKFYKL for the CPP (SEQ ID NO: 11) comprising the genetic engineering elements and the sequence MDmut2 in fusion with the protein MDA-7 (SEQ ID NO: 20).
SEQ ID NO: 49 which corresponds to the sequence ID NO: MHHHHHHHHENLYFQSGALGLPRREKNRVAARKRAKFKNLLQHYREVAAAKSSEN DRLRLLLKLENFQQRLQSLWTLARPFCPPLLATASQMQMVVLPCLGFTLLLWSQVS GAQGQEFHFGPCQVKGVVPQKLWEAFWAVKDTMQAQDNNTSCRLLQQEGLQNV SDAESCYLVHTLLEFYLKTVFKNYHNRTVEVRTLKSFSTLANNFVLIVSQLQPSQEN EMFSIRDSAHRRFLLFRRAFKQLDVEAALTKALGEVDILLTWMQKFYKL for the CPP (SEQ ID NO: 12) comprising the genetic engineering elements and the sequence MDmut2DelCys in fusion with the protein MDA-7 (SEQ ID NO: 20).
SEQ ID NO: 50 which corresponds to the sequence MHHHHHHHHENLYFQSGALGLPKRYKNRVASRKCRAKFKQAETQKLISEIDLLRKQ NEQLKHKLEQLLENFQQRLQSLWTLARPFCPPLLATASQMQMVVLPCLGFTLLLWS QVSGAQGQEFHFGPCQVKGVVPQKLWEAFWAVKDTMQAQDNNTSCRLLQQEGL QNVSDAESCYLVHTLLEFYLKTVFKNYHNRTVEVRTLKSFSTLANNFVLIVSQLQPS QENEMFSIRDSAHRRFLLFRRAFKQLDVEAALTKALGEVDILLTWMQKFYKL for the CPP (SEQ ID NO: 13) comprising the genetic engineering elements and the sequence MDmut3 in fusion with the protein MDA-7 (SEQ ID NO: 20).
SEQ ID NO: 51 which corresponds to the sequence MHHHHHHHHENLYFQSGALGLPKRYKNRVASRKRAKFKQAETQKLISEIDLLRKQN EQLKHKLEQLLENFQQRLQSLWTLARPFCPPLLATASQMQMVVLPCLGFTLLLWSQ VSGAQGQEFHFGPCQVKGVVPQKLWEAFWAVKDTMQAQDNNTSCRLLQQEGLQ NVSDAESCYLVHTLLEFYLKTVFKNYHNRTVEVRTLKSFSTLANNFVLIVSQLQPSQ ENEMFSIRDSAHRRFLLFRRAFKQLDVEAALTKALGEVDILLTWMQKFYKL for the CPP (SEQ ID NO: 14) comprising the genetic engineering elements and the sequence MDmut3DelCys in fusion with the protein MDA-7 (SEQ ID NO: 20).
SEQ ID NO: 52 which corresponds to the sequence MHHHHHHHHENLYFQSGALGLPRREKNRVAARKCRAKFKNAETQKLISEIDLLRKQ NEQLKHKLEQLLENFQQRLQSLWTLARPFCPPLLATASQMQMVVLPCLGFTLLLWS QVSGAQGQEFHFGPCQVKGVVPQKLWEAFWAVKDTMQAQDNNTSCRLLQQEGL QNVSDAESCYLVHTLLEFYLKTVFKNYHNRTVEVRTLKSFSTLANNFVLIVSQLQPS QENEMFSIRDSAHRRFLLFRRAFKQLDVEAALTKALGEVDILLTWMQKFYKL for the CPP (SEQ ID NO: 15) comprising the genetic engineering elements and the sequence MDmut4 in fusion with the protein MDA-7 (SEQ ID NO: 20).
SEQ ID NO: 53 which corresponds to the sequence MHHHHHHHHENLYFQSGALGLPRREKNRVAARKRAKFKNAETQKLISEIDLLRKQN EQLKHKLEQLLENFQQRLQSLWTLARPFCPPLLATASQMQMVVLPCLGFTLLLWSQ VSGAQGQEFHFGPCQVKGVVPQKLWEAFWAVKDTMQAQDNNTSCRLLQQEGLQ NVSDAESCYLVHTLLEFYLKTVFKNYHNRTVEVRTLKSFSTLANNFVLIVSQLQPSQ ENEMFSIRDSAHRRFLLFRRAFKQLDVEAALTKALGEVDILLTWMQKFYKL for the CPP (SEQ ID NO: 16) comprising the genetic engineering elements and the sequence MDmut4DelCys in fusion with the protein MDA-7 (SEQ ID NO: 20).
SEQ ID NO: 54 which corresponds to the sequence MHHHHHHHHENLYFQSGALGLPKRYKNRVASRKCRAKFKQLLQHYREVAAAKSSE NDRLRLLLKQLEGQEFHFGPCQVKGVVPQKLWEAFWAVKDTMQAQDNITSARLLQ QEVLQNVSDAESCYLVHTLLEFYLKTVFKNHHNRTVEVRTLKSFSTLANNFVLIVSQL QPSQENEMFSIRDSAHRRFLLFRRAFKQLDVEAALTKALGEVDILLTWMQKFYKL for the CPP (SEQ ID NO: 9) comprising the genetic engineering elements and the sequence MD11 in fusion with the truncated MDA-7 protein (SEQ ID NO: 21).
SEQ ID NO: 55 which corresponds to the sequence MMHHHHHHHHENLYFQSGALGLPKRYKNRVASRKRAKFKQLLQHYREVAAAKSSE NDRLRLLLKLEGQEFHFGPCQVKGVVPQKLWEAFWAVKDTMQAQDNITSARLLQQ EVLQNVSDAESCYLVHTLLEFYLKTVFKNHHNRTVEVRTLKSFSTLANNFVLIVSQLQ PSQENEMFSIRDSAHRRFLLFRRAFKQLDVEAALTKALGEVDILLTWMQKFYKL for the CPP (SEQ ID NO: 10) comprising the genetic engineering elements and the sequence MDmut1DelCys in fusion with the truncated MDA-7 protein (SEQ ID NO: 21).
SEQ ID NO: 56 which corresponds to the sequence MHHHHHHHHENLYFQSGALGLPRREKNRVAARKCRAKFKNLLQHYREVAAAKSSE NDRLRLLLKLEGQEFHFGPCQVKGVVPQKLWEAFWAVKDTMQAQDNITSARLLQQ EVLQNVSDAESCYLVHTLLEFYLKTVFKNHHNRTVEVRTLKSFSTLANNFVLIVSQLQ PSQENEMFSIRDSAHRRFLLFRRAFKQLDVEAALTKALGEVDILLTWMQKFYKL for the CPP (SEQ ID NO: 11) comprising the genetic engineering elements and the sequence MDmut2 in fusion with the truncated MDA-7 protein (SEQ ID NO: 21).
SEQ ID NO: 57 which corresponds to the sequence MHHHHHHHHENLYFQSGALGLPRREKNRVAARKRAKFKNLLQHYREVAAAKSSEN DRLRLLLKLEGQEFHFGPCQVKGVVPQKLWEAFWAVKDTMQAQDNITSARLLQQE VLQNVSDAESCYLVHTLLEFYLKTVFKNHHNRTVEVRTLKSFSTLANNFVLIVSQLQP SQENEMFSIRDSAHRRFLLFRRAFKQLDVEAALTKALGEVDILLTWMQKFYKL for the CPP (SEQ ID NO: 12) comprising the genetic engineering elements and the sequence MDmut2DelCys in fusion with the truncated MDA-7 protein (SEQ ID NO: 21).
SEQ ID NO: 58 which corresponds to the sequence MHHHHHHHHENLYFQSGALGLPKRYKNRVASRKCRAKFKQAETQKLISEIDLLRKQ NEQLKHKLEQLLEGQEFHFGPCQVKGVVPQKLWEAFWAVKDTMQAQDNITSARLL QQEVLQNVSDAESCYLVHTLLEFYLKTVFKNHHNRTVEVRTLKSFSTLANNFVLIVS QLQPSQENEMFSIRDSAHRRFLLFRRAFKQLDVEAALTKALGEVDILLTWMQKFYKL for the CPP (SEQ ID NO: 13) comprising the genetic engineering elements and the sequence MDmut3 in fusion with the truncated MDA-7 protein (SEQ ID NO: 21).
SEQ ID NO: 59 which corresponds to the sequence ID NO: MHHHHHHHHENLYFQSGALGLPKRYKNRVASRKRAKFKQAETQKLISEIDLLRKQN EQLKHKLEQLLEGQEFHFGPCQVKGVVPQKLWEAFWAVKDTMQAQDNITSARLLQ QEVLQNVSDAESCYLVHTLLEFYLKTVFKNHHNRTVEVRTLKSFSTLANNFVLIVSQL QPSQENEMFSIRDSAHRRFLLFRRAFKQLDVEAALTKALGEVDILLTWMQKFYKL for the CPP (SEQ ID NO: 14) comprising the genetic engineering elements and the sequence MDmut3DelCys in fusion with the truncated MDA-7 protein (SEQ ID NO: 21).
SEQ ID NO: 60 which corresponds to the sequence MHHHHHHHHENLYFQSGALGLPRREKNRVAARKCRAKFKNAETQKLISEIDLLRKQ NEQLKHKLEQLLEGQEFHFGPCQVKGVVPQKLWEAFWAVKDTMQAQDNITSARLL QQEVLQNVSDAESCYLVHTLLEFYLKTVFKNHHNRTVEVRTLKSFSTLANNFVLIVS QLQPSQENEMFSIRDSAHRRFLLFRRAFKQLDVEAALTKALGEVDILLTWMQKFYKL for the CPP (SEQ ID NO: 15) comprising the genetic engineering elements and the sequence MDmut4 in fusion with the truncated MDA-7 protein (SEQ ID NO: 21).
SEQ ID NO: 61 which corresponds to the sequence MHHHHHHHHENLYFQSGALGLPRREKNRVAARKRAKFKNAETQKLISEIDLLRKQN EQLKHKLEQLLEGQEFHFGPCQVKGVVPQKLWEAFWAVKDTMQAQDNITSARLLQ QEVLQNVSDAESCYLVHTLLEFYLKTVFKNHHNRTVEVRTLKSFSTLANNFVLIVSQL QPSQENEMFSIRDSAHRRFLLFRRAFKQLDVEAALTKALGEVDILLTWMQKFYKL for the CPP (SEQ ID NO: 16) comprising the genetic engineering elements and the sequence MDmut4DelCys in fusion with the truncated MDA-7 protein (SEQ ID NO: 21).

### Protocols for Producing and Purifying the Fusion Proteins

The protocols hereinafter have been developed by the “trial-and-error” technique on each of the following parameters:
Production: E. coli bacterial strain, time, temperature, culture volume, stress condition (ethanol addition at 1, 3 or 5%), induction optical density of the production, inducer concentration of the production (IPTG, isopropyl β-D-1-thiogalactopyranoside);
Lysis: chemical and/or mechanical method, time, power, repetition;
Purification: solubilization or not, buffer, affinity, purity, column type, renaturation conditions, dialysis.
   Production of the Fusion Proteins Comprising a CPP and the eGFP Polypeptide

The optimum conditions for producing and purifying the fusion proteins comprising a mutant CPP (MDmut) or MD11 and the eGFP polypeptide (SEQ ID NO: 22 to SEQ ID NO: 29) have been defined as described hereinafter.

The best production yield has been observed after culture of E. coli BL21 (DE3) bacteria transformed by the recombinant plasmids encoding the sequences SEQ ID NO: 22 to SEQ ID NO: 29 overnight at 16° C. after induction of the production with 0.5 mM IPGT added at DO260=1.

The lysis of the bacteria has been optimum twice in a Tris-NaCl-imidazole buffer. First of all, a chemical lysis with lysozyme at 1 mg/ml for 30 min on a wheel for a gentle stirring has been carried out at 4° C. in order to preserve the integrity of the proteins. Then, a mechanical lysis by 6 cycles of 30 sec sonication fore 3 cycles at 30% power and then 3 cycles at 70% power have followed, the bacterial lysates having been reset in ice at least for 30 sec between each sonication.

After centrifugation, the soluble fraction has been passed over a Nickel column and the proteins were able to be eluted by competition with 500 mM imidazole.

The purity, the quality and the quantity of the proteins have been analyzed by SDS-PAGE and Western blot. For this purpose, 15 μl of sample are mixed with 5 μl of 4× reducing buffer and migrated into a 12% acrylamide gel for 1 h at 30 mA-150V. The gels are either stained with Coomassie blue for 2 h at room temperature (TA) under stirring and then bleached with water, or transferred onto a 0.2 μm nitrocellulose membrane by the Bio-Rad Mini-protean system according to the protocol of the manufacturer on the “Mixed Molecular Weight” program. Afterwards, the membranes have been blocked with 5% skimmed milk-TBS 1× tween 0.1% for 2 h at TA under stirring and then incubated with an anti-His HRP (standing for “HorseRadish Peroxidase”) antibody diluted at 1/50,000 in the blocking solution. The anti-His antibody binds to the poly-histidine tag of the fusion proteins and the HRP enables detection thereof with the ChemiDoc Imaging System (BioRad) by chemiluminescence.

The Coomassie blue staining of the acrylamide gels has allowed us to notice that the higher the production yield, the less protein solubilization is effective. However, the amounts of solubilized proteins remain very large and sufficient for the purification of several milligrams of proteins according to the protocol.

In turn, the Western-blot has indicated to us the presence of protein aggregates in the samples that have been able to be removed by adding a very high-speed centrifugation step.

Production of the Fusion Proteins Comprising a CCP and the Polypeptide elF3f

The optimum conditions for producing and purifying the fusion proteins comprising a mutant CPP (MDmut) or MD11 and the polypeptide elF3f (SEQ ID NO: 30 to SEQ ID NO: 37) are defined hereinafter.

The best production yield has been observed after culture of E. coli BL21 (DE3) bacteria transformed by the recombinant plasmids encoding the sequences SEQ ID NO: 30 to SEQ ID NO: 37 overnight at 16° C. after induction of the production at 0.5 mM of IPGT added at DO=1.

The lysis of the bacteria has been optimum in 2 times in a MOPS-NaCl-imidazole buffer. First, a chemical lysis with lysozyme at 1 mg/ml for 30 min on a wheel for a gentle stirring has been carried out at 4° C. in order to preserve the integrity of the proteins. Then, a mechanical lysis by 5 cycles of 30 sec sonication at 70% power has followed, the bacterial lysates having been reset in ice at least for 30 sec between each sonication.

None of the tested protocols allowed obtaining a sufficient amount of proteins in soluble form. After centrifugation, the soluble fraction has therefore been removed and the inclusion bodies have been washed in 3 different washing buffers (MOPS 20 mM-NaCl 2M-Cysteine 15 mM; MOPS 20 mM-NaCl 250 mM-Cysteine 15 mM-Tritton X100 2%; MOPS 20 mM-NaCl 250 mM-Cysteine 15 mM) and then solubilized under denaturing conditions (MOPS 20 mM-NaCl 250 mM-Cysteine 15 mM-Urea 8M). The proteins have been purified on a nickel column by imidazole competition (MOPS 20 mM-NaCl 250 mM-Cysteine 15 mM-Urea 8M-Imidazole 500 mM), the proteins have been renaturated by the dropwise dilution method (Na Citrate 50 mM-MgCl2 2 mM-DTT 2 mM-NaCl 50 mM-L-Argine 500 mM-Tween 20 0.5%) and then dialyzed twice (Na Citrate 50 mM-MgCl2 2 mM-DTT 2 mM-NaCl 50 mM-L-Argine 50 mM-N-Lauryl Sarcosine 0.01%; Na Citrate 50 mM-NaCl 50 mM-Glycerol 10%) to be set in solution in a buffer compatible with in cellulo and in vivo experiments.

The purity and quality of the proteins have been analyzed by SDS-PAGE and Western blot with the same protocol as that used for the fusion proteins comprising the eGFP protein and described in the previous section. In turn, the treatment samples have been assayed by the microBCA method according to the protocol of the manufacturer.

In the elution samples, refolding samples and dialysates, one single 50 kDa band, labeled with the anti-histidine antibody in Western blot, has been highlighted by staining the acrylamide gel with Coomassie blue. Hence, the treatment samples used for the in vitro experiments are pure and of good quality. Indeed, the absence of a supernumerary band in Western blot has demonstrated the absence of any degraded and/or aggregated protein in the samples.

### Production of the Fusion Proteins Comprising a CCP and the MDA-7 or Truncated MDA-7 Polypeptide

The optimum conditions for producing and purifying the fusion proteins comprising a mutant CPP (MDmut) or MD11 and the MDA-7 polypeptide (SEQ ID NO: 46 to SEQ ID NO: 53) or the truncated MDA-7 polypeptide (SEQ ID NO: 54 to SEQ ID NO: 61) are defined hereinafter.

The best production yield has been observed after culture of E. coli BL21 (DE3) bacteria transformed by the recombinant plasmids encoding the sequences SEQ ID NO: 46 to SEQ ID NO: 61 overnight at 16° C. after induction of the production with 1M of IPGT added at DO260>1.

The lysis of the bacteria has been optimum twice in a Tris 50 mM-NaCl 250 mM-imidazole 5 mM buffer. First of all, a chemical lysis with lysozyme at 1 mg/ml for 30 min on a wheel for a gentle stirring has been carried out at 4° C. in order to preserve the integrity of the proteins. Then, a mechanical lysis by 5 cycles of 30 sec sonication at 70% power has followed, the bacterial lysates having been reset in ice at least for 30 sec between each sonication.

None of the tested protocols allowed obtaining a sufficient concentration of proteins in soluble form. After centrifugation, the soluble fraction has therefore been removed and the inclusion bodies have been washed in 3 different wash buffers (Tris 50 mM-β-mercaptoethanol 20 mM-Tritton X100 2%; Tris 20 mM-β-mercaptoethanol 20 mM; Tris 50 mM-DTT 20 mM-Guanidine HydroChloride 6M) and then solubilized under Guanidine denaturing conditions. The proteins have been purified on a Nickel column by imidazole competition (Tris 50 mM Guanidine HydroChloride 6M-Imidazole 500 mM) and renaturated by the dropwise dilution method (Tris 50 mM-Guanidine HydroChloride 1,4M-NaCl 50 mM-L-Argine 500 mM-Titton X100 0.5%) and then dialyzed twice in order to be set in solution (Tris 50 mM-Guanidine HydroChloride 0.75M-NaCl 50 mM-L-Argine 50 mM-Tween20 0.5%; Tris 50 mM-NaCl 50 mM-Glycerol 10%) in a buffer compatible with in cellulo and in vivo experiments.

The purity and the quality of the proteins have been analyzed by SDS-PAGE and Western blot and then these have been assayed by the microBCA method. One single band, labeled with the anti-histidine antibody in Western blot, has been highlighted by staining the acrylamide gel with Coomassie blue. Hence, the samples of CPP fusion proteins coupled to the whole or truncated MDA7 polypeptides are pure and of good quality. Indeed, the absence of a supernumerary band in Western blot has demonstrated the absence of degraded and/or aggregated protein in the samples.

### Analysis of the Cell-Penetrating Potential of the CPPs

In order to demonstrate the high intracellular-penetrating potential of the MDmut mutant CPP, flow cytometry and fluorescence microscopy analyzes have been carried out.

The treatments by the fusion proteins comprising a mutant CPP and the eGFP polypeptide (SEQ ID NO: 23 to SEQ ID NO: 29) have been compared to the treatment with the eGFP alone, not bound to a delivery system, and to the treatments by the fusion proteins comprising either the CPP MD11 (SEQ ID NO: 1) without the novel genetic engineering elements and the eGFP polypeptide (SEQ ID NO: 17), or comprising the CPP MD11 with the new genetic engineering elements and the eGFP polypeptide (SEQ ID NO: 22).

To count the number of fluorescent cells after treatment, HEK293, B16-Ova and beta-TC-6 cells have been seeded and then treated at about 60% confluence per 200 nM of respective fusion proteins in the absence of bovine serum for 4 h. Afterwards, they have been supplemented with 5% bovine serum and incubated overnight. The next day, the cells have been trypsinized and then washed with PBS 1× and passed to the FACS for analyzes.

An untreated cell sample supplemented with 1 μg/ml, has allowed calibrating the white and the living cell range. For the treated samples, a minimum of 25,000 live cells have been analyzed for their green fluorescence.

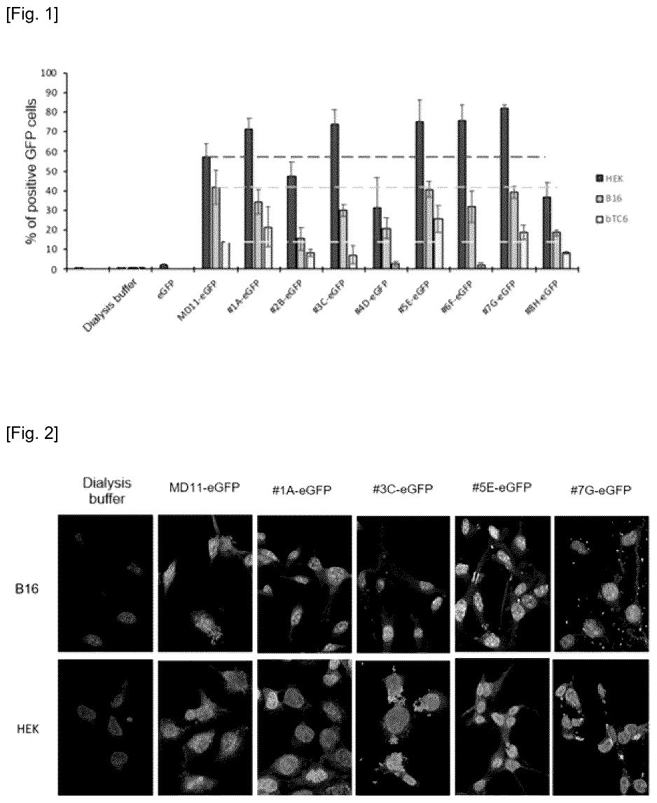

The analysis results indicate the percentage of positive GFP cells after treatment and are shown in FIG. 1 wherein:
MD11-eGFP corresponds to the control fusion protein comprising the sequence of the CPP MD11 but without the genetic engineering elements (SEQ ID NO: 1) and the eGFP polypeptide (SEQ ID NO: 17);
#1A-eGFP corresponds to the fusion protein comprising the sequence of the CPP MD11 with the genetic engineering elements and the eGFP polypeptide (SEQ ID NO: 22);
#2B-eGFP corresponds to the fusion protein comprising the sequence of the CPP MDmut1DelCys and the eGFP polypeptide (SEQ ID NO: 23);
#3C-eGFP corresponds to the fusion protein comprising the sequence of the CPP MDmut2 and the eGFP polypeptide (SEQ ID NO: 24);
#4D-eGFP corresponds to the fusion protein comprising the sequence of the CPP MDmut2DelCys and the eGFP polypeptide (SEQ ID NO: 25);
#5E-eGFP corresponds to the fusion protein comprising the sequence of CPP MDmut3 and the eGFP polypeptide (SEQ ID NO: 26);
#6F-eGFP corresponds to the fusion protein comprising the sequence of the CPP MDmut3DelCys and the eGFP polypeptide (SEQ ID NO: 27);
#7G-eGFP corresponds to the fusion protein comprising the sequence of CPP MDmut4 and the eGFP polypeptide (SEQ ID NO: 28);
#8H-eGFP corresponds to the fusion protein comprising the sequence of the CPP MDmut4DelCys and the eGFP polypeptide (SEQ ID NO: 29).

Flow cytometry has shown that, in general, under current treatment conditions with a replicas number comprised between 3 and 5 per condition, the penetration power of the proteins is higher in the HEK293 human non-tumor cells than in the B16-Ova tumor cells (B16) and the beta-TC-6 human tumor cells (bTC6). For the MD11-eGFP and #5E-eGFP fusion proteins, we are at the limit of the significance p=0.057. The same penetration defect, with respect to MD11-eGFP, is observed for the #4D-eGFP mutant in the three cell lines.

In HEK293 cells, the fusion proteins comprising a mutant CPP and the eGFP polypeptide have a preserved cell-penetrating capacity, including an increasing non-significant trend for #3C-eGFP, #5E-eGFP, #6F-eGFP and #7G-eGFP compared to the control (MD11-eGFP) with a GFP-positive cell rate comprised between 73% and 82% against 57%.

In B16-Ova cells (B16), the penetration capacity of the mutants #3C-eGFP, #5E-eGFP, #6F-eGFP and #7G-eGFP is equivalent to that of MD11-eGFP. On the other hand, the cell-penetrating capacity of the mutants #2B-eGFP, #4D-eGFP and #8H-eGFP is significantly lower between 15% and 20% against more than 41%.

In bTC6 cells, the penetration capacity of the mutants #1A-eGFP, #5E-eGFP and #7G-eGFP is significantly higher between 21% and 30% against 15% for MD11-eGFP. On the other hand, the penetration capacity of the mutants #2B-eGFP, #3C-eGFP, #4D-eGFP, #6F-eGFP and #8H-eGFP is significantly lower between 1% and 8% against 15%.

In parallel, the same cells (HEK293 and B16-Ova) have also been seeded on glass slides equipped with culture chambers and then treated with the MD11-eGFP, #1A-eGFP, #3C-eGFP, #5E-eGFP, #7G-eGFP fusion proteins or the pdt 4 h buffer in the absence of serum. Afterwards, the cells have been washed with PBS 1× and then with heparin and bonded to Paraformaldehyde and observed under a fluorescence microscope. 5 successive focal planes spaced apart by 2 μm have been captured in order to highlight the intracellular localization of the proteins.

The images representative of the fluorescence of the cells after treatment are given in FIG. 2.

The cell-penetrating efficiency of the different mutants being unequal, some fluorescent signals have been “forced” to be visible but all have been identified as intracellular. However, the protein aggregates visualized on some images seem to be bonded to the membranes.

Analysis of the Anti-Tumor Therapeutic Potential of the Fusion Protein Comprising CPP and the Polypeptide elF3f

In order to demonstrate the therapeutic potential of the MDmut mutant CPPs fused to the translation regulatory factor elF3f (SEQ ID NO: 31 to SEQ ID NO: 37) on melanoma, the study is carried on still with the HEK293 and B16-Ova cells.

The viability has been tested with Prestobia®. For this purpose, the cells have been seeded in 96-well plates with black edges and transparent bottoms and then, the next day, treated at 60% confluence with 0.25× fetal calf serum (FCS). In order to calculate the median inhibitory concentration (IC50) of each fusion protein, these have been diluted according to a wide concentration range comprised between 15 and 100 nM and incubated for 24 h or 48 h.

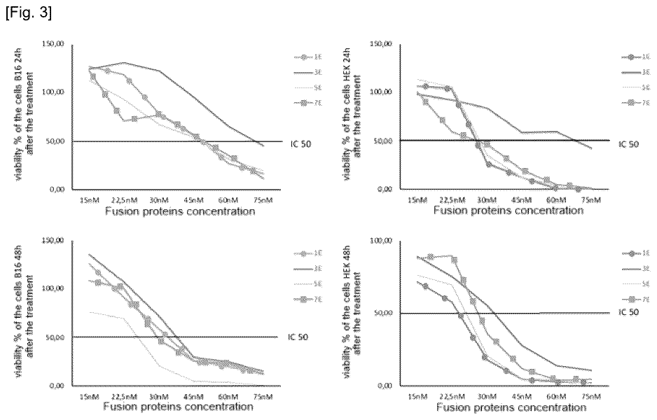

The viability curves of the cells B16-Ova (B16) and HEK293 after 24 h and 48 h of treatment by the fusion proteins are illustrated in FIG. 3 wherein:
1E corresponds to the fusion protein comprising the sequence of CPP MD11 and the polypeptide elF3f (SEQ ID NO: 30);
3E corresponds to the fusion protein comprising the sequence of CPP MDmut2 and the polypeptide elF3f (SEQ ID NO: 32);
5E corresponds to the fusion protein comprising the sequence of CPP MDmut3 and the polypeptide elF3f (SEQ ID NO: 34);
7E corresponds to the fusion protein comprising the sequence of CPP MDmut4 and the polypeptide elF3f (SEQ ID NO: 36).

These tests have shown that the 3E mutant CPP proves to be the least effective both on B16 cells and HEK293 after 24 or 48 h. The IC50 at 24 h of the other mutant CPP is the same as about 50 nM for the B16 cells and 30 nM for the HEK293 cells. The IC50 at 48 h is not significantly different either and is between 25 and 35 nM, an extremely low concentration which corroborates a direct and intermediate action of the fusion proteins in the cells.

These tests have also showed that the fusion proteins #1A-eGFP, #5E-eGFP and #7G-eGFP (and by extension the mutant peptides that form them) have a better cell-penetrating capacity than that of the fusion protein formed with MD11. This increased capacity has been confirmed in at least two different cell lines, in this case the non-tumor human line HEK and the tumor human line bTC6.

More generally, it should be noted that the implementations and embodiments of the invention considered hereinabove have been described as non-limiting examples and that other variants could consequently be considered.

1. A peptide comprising or consisting of an amino acid sequence selected from among the sequences SEQ ID NO: 2, SEQ ID NO: 3, SEQ ID NO: 4, SEQ ID NO: 5, SEQ ID NO: 6, SEQ ID NO: 7 and SEQ ID NO: 8.

2. A nucleic acid molecule encoding a peptide according to claim 1.

3. An expression vector comprising a nucleic acid molecule encoding a peptide according to claim 1.

4. A host cell comprising a nucleic acid molecule according to claim 2 or an expression vector comprising a nucleic acid molecule encoding a peptide.

5. A method for utilizing a peptide according to claim 1 or of a nucleic acid molecule encoding the peptide or of an expression vector comprising a nucleic acid molecule encoding the peptide or of a host cell comprising the nucleic acid molecule or the expression vector, for obtaining a delivery system intended for the internalization of a molecule of interest in target cells.

6. A combination comprising a delivery system and a molecule of interest, the delivery system being a peptide according to claim 1.

7. A fusion protein comprising a delivery system which is a peptide according to claim 1, and a molecule of interest which is a polypeptide of interest.

8. A nucleic acid molecule encoding the fusion protein according to claim 7.

9. An expression vector comprising a nucleic acid molecule encoding the fusion protein according to claim 7.

10. A host cell comprising a nucleic acid molecule according to claim 8 or an expression vector comprising a nucleic acid molecule encoding the fusion protein.

11. A pharmaceutical composition comprising a combination according to claim 6 or a fusion protein comprising a delivery system which is a peptide comprising or consisting of an amino acid sequence selected from among the sequences SEQ ID NO: 2, SEQ ID NO: 3, SEQ ID NO: 4, SEQ ID NO: 5, SEQ ID NO: 6, SEQ ID NO: 7 and SEQ ID NO: 8, and a molecule of interest which is a polypeptide of interest, as well as a pharmaceutically-acceptable excipient and/or carrier.

12. The combination according to claim 6 or the fusion protein comprising a delivery system which is a peptide comprising or consisting of an amino acid sequence selected from among the sequences SEQ ID NO: 2, SEQ ID NO: 3, SEQ ID NO: 4, SEQ ID NO: 5, SEQ ID NO: 6, SEQ ID NO: 7 and SEQ ID NO: 8, and a molecule of interest which is a polypeptide of interest, or the pharmaceutical composition comprising a combination according to claim 6 or the fusion protein, as well as a pharmaceutically-acceptable excipient and/or carrier, for use in the treatment, diagnosis or prevention of pathologies, including cancers.

13. The combination according to claim 6, the fusion protein comprising a delivery system which is a peptide comprising or consisting of an amino acid sequence selected from among the sequences SEQ ID NO: 2, SEQ ID NO: 3, SEQ ID NO: 4, SEQ ID NO: 5, SEQ ID NO: 6, SEQ ID NO: 7 and SEQ ID NO: 8, and a molecule of interest which is a polypeptide of interest, the nucleic acid molecule encoding the fusion protein, the expression vector comprising a nucleic acid molecule encoding the fusion protein, the host cell comprising the nucleic acid module or the expression vector, or the pharmaceutical composition comprising a combination according to claim 6 or the fusion protein, as well as a pharmaceutically-acceptable excipient and/or carrier, wherein the molecule of interest comprises or consists of a polypeptide of interest selected from among a polypeptide encoding the eGFP protein represented by the sequence SEQ ID NO: 17, a polypeptide encoding the elF3f protein represented by the sequence SEQ ID NO: 18, a polypeptide encoding the FERM protein represented by the sequence SEQ ID NO: 19, a polypeptide encoding the MDA-7 protein represented by the sequence SEQ ID NO: 20, a polypeptide encoding the truncated MDA-7 protein represented by the sequence SEQ ID NO: 21 and a polypeptide represented by a sequence having 80% sequence identity with one of the sequences SEQ ID NO: 17, SEQ ID NO: 18, SEQ ID NO: 19, SEQ ID NO: 20 and SEQ ID NO: 21.

14. The combination or fusion protein or pharmaceutical composition for use according to claim 12, wherein the molecule of interest comprises or consists of a polypeptide of interest selected from among a polypeptide encoding the eGFP protein represented by the sequence SEQ ID NO: 17, a polypeptide encoding the elF3f protein represented by the sequence SEQ ID NO: 18, a polypeptide encoding the FERM protein represented by the sequence SEQ ID NO: 19, a polypeptide encoding the MDA-7 protein represented by the sequence SEQ ID NO: 20, a polypeptide encoding the truncated MDA-7 protein represented by the sequence SEQ ID NO: 21 and a polypeptide represented by a sequence having 80% sequence identity with one of the sequences SEQ ID NO: 17, SEQ ID NO: 18, SEQ ID NO: 19, SEQ ID NO: 20 and SEQ ID NO: 21.

15. The fusion protein according to claim 7, comprising or consisting of an amino acid sequence selected from among SEQ ID NO: 23 to SEQ ID NO: 29, SEQ ID NO: 31 to SEQ ID NO: 37, SEQ ID NO: 39 to SEQ ID NO: 45, SEQ ID NO: 47 to SEQ ID NO: 53 and SEQ ID NO: 55 to SEQ ID NO: 61.


SEQUENCE LISTING (61 sequences)

SEQ ID NO: 1 - protein, 43 aa, Epstein-Barr virus
KRYKNRVASRKCRAKFKQLLQHYREVAAAKSSENDRLRLLLKQ

SEQ ID NO: 2 - protein, 41 aa, synthetic construct
KRYKNRVASRKRAKFKQLLQHYREVAAAKSSENDRLRLLLK

SEQ ID NO: 3 - protein, 42 aa, synthetic construct
RREKNRVAARKCRAKFKNLLQHYREVAAAKSSENDRLRLLLK

SEQ ID NO: 4 - protein, 41 aa, synthetic construct
RREKNRVAARKRAKFKNLLQHYREVAAAKSSENDRLRLLLK

SEQ ID NO: 5 - protein, 45 aa, synthetic construct
KRYKNRVASRKCRAKFKQAETQKLISEIDLLRKQNEQLKHKLEQL

SEQ ID NO: 6 - protein, 44 aa, synthetic construct
KRYKNRVASRKRAKFKQAETQKLISEIDLLRKQNEQLKHKLEQL

SEQ ID NO: 7 - protein, 45 aa, synthetic construct
RREKNRVAARKCRAKFKNAETQKLISEIDLLRKQNEQLKHKLEQL

SEQ ID NO: 8 - protein, 44 aa, synthetic construct
RREKNRVAARKRAKFKNAETQKLISEIDLLRKQNEQLKHKLEQL

SEQ ID NO: 9 - protein, 65 aa, synthetic construct
MHHHHHHHHENLYFQSGALGLPKRYKNRVASRKCRAKFKQLLQHYREVAAAKSSENDRLRLLLKQ

SEQ ID NO: 10 - protein, 63 aa, synthetic construct
MHHHHHHHHENLYFQSGALGLPKRYKNRVASRKRAKFKQLLQHYREVAAAKSSENDRLRLLLK

SEQ ID NO: 11 - protein, 64 aa, synthetic construct
MHHHHHHHHENLYFQSGALGLPRREKNRVAARKCRAKFKNLLQHYREVAAAKSSENDRLRLLLK

SEQ ID NO: 12 - protein, 63 aa, synthetic construct
MHHHHHHHHENLYFQSGALGLPRREKNRVAARKRAKFKNLLQHYREVAAAKSSENDRLRLLLK

SEQ ID NO: 13 - protein, 67 aa, synthetic construct
MHHHHHHHHENLYFQSGALGLPKRYKNRVASRKCRAKFKQAETQKLISEIDLLRKQNEQLKHKLEQL

SEQ ID NO: 14 - protein, 66 aa, synthetic construct
MHHHHHHHHENLYFQSGALGLPKRYKNRVASRKRAKFKQAETQKLISEIDLLRKQNEQLKHKLEQL

SEQ ID NO: 15 - protein, 67 aa, synthetic construct
MHHHHHHHHENLYFQSGALGLPRREKNRVAARKCRAKFKNAETQKLISEIDLLRKQNEQLKHKLEQL

SEQ ID NO: 16 - protein, 66 aa, synthetic construct
MHHHHHHHHENLYFQSGALGLPRREKNRVAARKRAKFKNAETQKLISEIDLLRKQNEQLKHKLEQL

SEQ ID NO: 17 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 18 - protein, 356 aa, Homo sapiens
ATPAVPVSAPPATPTPVPAAAPASVPAPTPAPAAAPVPAAAPASSSDPAAAAAATAAPGQTPASAQAPAQTPAPALPGPALPGPFPGGRVVRLHPVILASIVDSYERRNEGAARVIGTLLGTVDKHSVEVTNCFSVPHNESEDEVAVDMEFAKNMYELHKKVSPNELILGWYATGHDITEHSVLIHEYYSREAPNPIHLTVDTSLQNGRMSIKAYVSTLMGVPGRTMGVMFTPLTVKYAYYDTERIGVDLIMKTCFSPNRVIGLSSDLQQVGGASARIQDALSTVLQYAEDVLSGKVSADNTVGRFLMSLVNQVPKIVPDDFETMLNSNINDLLMVTYLANLTQSQIALNEKLVNL

SEQ ID NO: 19 - protein, 320 aa, Homo sapiens
MPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLER

SEQ ID NO: 20 - protein, 205 aa, Homo sapiens
NFQQRLQSLWTLARPFCPPLLATASQMQMVVLPCLGFTLLLWSQVSGAQGQEFHFGPCQVKGVVPQKLWEAFWAVKDTMQAQDNNTSCRLLQQEGLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 21 - protein, 156 aa, Homo sapiens
GQEFHFGPCQVKGVVPQKLWEAFWAVKDTMQAQDNITSARLLQQEVLQNVSDAESCYLVHTLLEFYLKTVFKNHHNRTVEVRTLKSFSTLANNFVLIVSQLQPSQENEMFSIRDSAHRRFLLFRRAFKQLDVEAALTKALGEVDILLTWMQKFYKL

SEQ ID NO: 22 - protein, 306 aa, synthetic construct
MHHHHHHHHENLYFQSGALGLPKRYKNRVASRKCRAKFKQLLQHYREVAAAKSSENDRLRLLLKQLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 23 - protein, 304 aa, synthetic construct
MHHHHHHHHENLYFQSGALGLPKRYKNRVASRKRAKFKQLLQHYREVAAAKSSENDRLRLLLKLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 24 - protein, 305 aa, synthetic construct
MHHHHHHHHENLYFQSGALGLPRREKNRVAARKCRAKFKNLLQHYREVAAAKSSENDRLRLLLKLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 25 - protein, 304 aa, synthetic construct
MHHHHHHHHENLYFQSGALGLPRREKNRVAARKRAKFKNLLQHYREVAAAKSSENDRLRLLLKLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 26 - protein, 308 aa, synthetic construct
MHHHHHHHHENLYFQSGALGLPKRYKNRVASRKCRAKFKQAETQKLISEIDLLRKQNEQLKHKLEQLLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 27 - protein, 307 aa, synthetic construct
MHHHHHHHHENLYFQSGALGLPKRYKNRVASRKRAKFKQAETQKLISEIDLLRKQNEQLKHKLEQLLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 28 - protein, 308 aa, synthetic construct
MHHHHHHHHENLYFQSGALGLPRREKNRVAARKCRAKFKNAETQKLISEIDLLRKQNEQLKHKLEQLLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 29 - protein, 307 aa, synthetic construct
MHHHHHHHHENLYFQSGALGLPRREKNRVAARKRAKFKNAETQKLISEIDLLRKQNEQLKHKLEQLLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 30 - protein, 423 aa, synthetic construct
MHHHHHHHHENLYFQSGALGLPKRYKNRVASRKCRAKFKQLLQHYREVAAAKSSENDRLRLLLKQLEATPAVPVSAPPATPTPVPAAAPASVPAPTPAPAAAPVPAAAPASSSDPAAAAAATAAPGQTPASAQAPAQTPAPALPGPALPGPFPGGRVVRLHPVILASIVDSYERRNEGAARVIGTLLGTVDKHSVEVTNCFSVPHNESEDEVAVDMEFAKNMYELHKKVSPNELILGWYATGHDITEHSVLIHEYYSREAPNPIHLTVDTSLQNGRMSIKAYVSTLMGVPGRTMGVMFTPLTVKYAYYDTERIGVDLIMKTCFSPNRVIGLSSDLQQVGGASARIQDALSTVLQYAEDVLSGKVSADNTVGRFLMSLVNQVPKIVPDDFETMLNSNINDLLMVTYLANLTQSQIALNEKLVNL

SEQ ID NO: 31 - protein, 421 aa, synthetic construct
MHHHHHHHHENLYFQSGALGLPKRYKNRVASRKRAKFKQLLQHYREVAAAKSSENDRLRLLLKLEATPAVPVSAPPATPTPVPAAAPASVPAPTPAPAAAPVPAAAPASSSDPAAAAAATAAPGQTPASAQAPAQTPAPALPGPALPGPFPGGRVVRLHPVILASIVDSYERRNEGAARVIGTLLGTVDKHSVEVTNCFSVPHNESEDEVAVDMEFAKNMYELHKKVSPNELILGWYATGHDITEHSVLIHEYYSREAPNPIHLTVDTSLQNGRMSIKAYVSTLMGVPGRTMGVMFTPLTVKYAYYDTERIGVDLIMKTCFSPNRVIGLSSDLQQVGGASARIQDALSTVLQYAEDVLSGKVSADNTVGRFLMSLVNQVPKIVPDDFETMLNSNINDLLMVTYLANLTQSQIALNEKLVNL

SEQ ID NO: 32 - protein, 422 aa, synthetic construct
MHHHHHHHHENLYFQSGALGLPRREKNRVAARKCRAKFKNLLQHYREVAAAKSSENDRLRLLLKLEATPAVPVSAPPATPTPVPAAAPASVPAPTPAPAAAPVPAAAPASSSDPAAAAAATAAPGQTPASAQAPAQTPAPALPGPALPGPFPGGRVVRLHPVILASIVDSYERRNEGAARVIGTLLGTVDKHSVEVTNCFSVPHNESEDEVAVDMEFAKNMYELHKKVSPNELILGWYATGHDITEHSVLIHEYYSREAPNPIHLTVDTSLQNGRMSIKAYVSTLMGVPGRTMGVMFTPLTVKYAYYDTERIGVDLIMKTCFSPNRVIGLSSDLQQVGGASARIQDALSTVLQYAEDVLSGKVSADNTVGRFLMSLVNQVPKIVPDDFETMLNSNINDLLMVTYLANLTQSQIALNEKLVNL

SEQ ID NO: 33 - protein, 421 aa, synthetic construct
MHHHHHHHHENLYFQSGALGLPRREKNRVAARKRAKFKNLLQHYREVAAAKSSENDRLRLLLKLEATPAVPVSAPPATPTPVPAAAPASVPAPTPAPAAAPVPAAAPASSSDPAAAAAATAAPGQTPASAQAPAQTPAPALPGPALPGPFPGGRVVRLHPVILASIVDSYERRNEGAARVIGTLLGTVDKHSVEVTNCFSVPHNESEDEVAVDMEFAKNMYELHKKVSPNELILGWYATGHDITEHSVLIHEYYSREAPNPIHLTVDTSLQNGRMSIKAYVSTLMGVPGRTMGVMFTPLTVKYAYYDTERIGVDLIMKTCFSPNRVIGLSSDLQQVGGASARIQDALSTVLQYAEDVLSGKVSADNTVGRFLMSLVNQVPKIVPDDFETMLNSNINDLLMVTYLANLTQSQIALNEKLVNL

SEQ ID NO: 34 - protein, 425 aa, synthetic construct
MHHHHHHHHENLYFQSGALGLPKRYKNRVASRKCRAKFKQAETQKLISEIDLLRKQNEQLKHKLEQLLEATPAVPVSAPPATPTPVPAAAPASVPAPTPAPAAAPVPAAAPASSSDPAAAAAATAAPGQTPASAQAPAQTPAPALPGPALPGPFPGGRVVRLHPVILASIVDSYERRNEGAARVIGTLLGTVDKHSVEVTNCFSVPHNESEDEVAVDMEFAKNMYELHKKVSPNELILGWYATGHDITEHSVLIHEYYSREAPNPIHLTVDTSLQNGRMSIKAYVSTLMGVPGRTMGVMFTPLTVKYAYYDTERIGVDLIMKTCFSPNRVIGLSSDLQQVGGASARIQDALSTVLQYAEDVLSGKVSADNTVGRFLMSLVNQVPKIVPDDFETMLNSNINDLLMVTYLANLTQSQIALNEKLVNL

SEQ ID NO: 35 - protein, 424 aa, synthetic construct
MHHHHHHHHENLYFQSGALGLPKRYKNRVASRKRAKFKQAETQKLISEIDLLRKQNEQLKHKLEQLLEATPAVPVSAPPATPTPVPAAAPASVPAPTPAPAAAPVPAAAPASSSDPAAAAAATAAPGQTPASAQAPAQTPAPALPGPALPGPFPGGRVVRLHPVILASIVDSYERRNEGAARVIGTLLGTVDKHSVEVTNCFSVPHNESEDEVAVDMEFAKNMYELHKKVSPNELILGWYATGHDITEHSVLIHEYYSREAPNPIHLTVDTSLQNGRMSIKAYVSTLMGVPGRTMGVMFTPLTVKYAYYDTERIGVDLIMKTCFSPNRVIGLSSDLQQVGGASARIQDALSTVLQYAEDVLSGKVSADNTVGRFLMSLVNQVPKIVPDDFETMLNSNINDLLMVTYLANLTQSQIALNEKLVNL

SEQ ID NO: 36 - protein, 425 aa, synthetic construct
MHHHHHHHHENLYFQSGALGLPRREKNRVAARKCRAKFKNAETQKLISEIDLLRKQNEQLKHKLEQLLEATPAVPVSAPPATPTPVPAAAPASVPAPTPAPAAAPVPAAAPASSSDPAAAAAATAAPGQTPASAQAPAQTPAPALPGPALPGPFPGGRVVRLHPVILASIVDSYERRNEGAARVIGTLLGTVDKHSVEVTNCFSVPHNESEDEVAVDMEFAKNMYELHKKVSPNELILGWYATGHDITEHSVLIHEYYSREAPNPIHLTVDTSLQNGRMSIKAYVSTLMGVPGRTMGVMFTPLTVKYAYYDTERIGVDLIMKTCFSPNRVIGLSSDLQQVGGASARIQDALSTVLQYAEDVLSGKVSADNTVGRFLMSLVNQVPKIVPDDFETMLNSNINDLLMVTYLANLTQSQIALNEKLVNL

SEQ ID NO: 37 - protein, 424 aa, synthetic construct
MHHHHHHHHENLYFQSGALGLPRREKNRVAARKRAKFKNAETQKLISEIDLLRKQNEQLKHKLEQLLEATPAVPVSAPPATPTPVPAAAPASVPAPTPAPAAAPVPAAAPASSSDPAAAAAATAAPGQTPASAQAPAQTPAPALPGPALPGPFPGGRVVRLHPVILASIVDSYERRNEGAARVIGTLLGTVDKHSVEVTNCFSVPHNESEDEVAVDMEFAKNMYELHKKVSPNELILGWYATGHDITEHSVLIHEYYSREAPNPIHLTVDTSLQNGRMSIKAYVSTLMGVPGRTMGVMFTPLTVKYAYYDTERIGVDLIMKTCFSPNRVIGLSSDLQQVGGASARIQDALSTVLQYAEDVLSGKVSADNTVGRFLMSLVNQVPKIVPDDFETMLNSNINDLLMVTYLANLTQSQIALNEKLVNL

SEQ ID NO: 38 - protein, 386 aa, synthetic construct
MHHHHHHHHENLYFQSGALGLPKRYKNRVASRKCRAKFKQLLQHYREVAAAKSSENDRLRLLLKQLEPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLER

SEQ ID NO: 39 - protein, 384 aa, synthetic construct
MHHHHHHHHENLYFQSGALGLPKRYKNRVASRKRAKFKQLLQHYREVAAAKSSENDRLRLLLKLEPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLER

SEQ ID NO: 40 - protein, 385 aa, synthetic construct
MHHHHHHHHENLYFQSGALGLPRREKNRVAARKCRAKFKNLLQHYREVAAAKSSENDRLRLLLKLEPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLER

SEQ ID NO: 41 - protein, 384 aa, synthetic construct
MHHHHHHHHENLYFQSGALGLPRREKNRVAARKRAKFKNLLQHYREVAAAKSSENDRLRLLLKLEPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLER

SEQ ID NO: 42 - protein, 388 aa, synthetic construct
MHHHHHHHHENLYFQSGALGLPKRYKNRVASRKCRAKFKQAETQKLISEIDLLRKQNEQLKHKLEQLLEPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLER

SEQ ID NO: 43 - protein, 387 aa, synthetic construct
MHHHHHHHHENLYFQSGALGLPKRYKNRVASRKRAKFKQAETQKLISEIDLLRKQNEQLKHKLEQLLEPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLER

SEQ ID NO: 44 - protein, 388 aa, synthetic construct
MHHHHHHHHENLYFQSGALGLPRREKNRVAARKCRAKFKNAETQKLISEIDLLRKQNEQLKHKLEQLLEPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLER

SEQ ID NO: 45 - protein, 388 aa, synthetic construct
MMHHHHHHHHENLYFQSGALGLPRREKNRVAARKRAKFKNAETQKLISEIDLLRKQNEQLKHKLEQLLEPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLER

SEQ ID NO: 46 - protein, 272 aa, synthetic construct
MHHHHHHHHENLYFQSGALGLPKRYKNRVASRKCRAKFKQLLQHYREVAAAKSSENDRLRLLLKQLENFQQRLQSLWTLARPFCPPLLATASQMQMVVLPCLGFTLLLWSQVSGAQGQEFHFGPCQVKGVVPQKLWEAFWAVKDTMQAQDNNTSCRLLQQEGLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 47 - protein, 270 aa, synthetic construct
MHHHHHHHHENLYFQSGALGLPKRYKNRVASRKRAKFKQLLQHYREVAAAKSSENDRLRLLLKLENFQQRLQSLWTLARPFCPPLLATASQMQMVVLPCLGFTLLLWSQVSGAQGQEFHFGPCQVKGVVPQKLWEAFWAVKDTMQAQDNNTSCRLLQQEGLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 48 - protein, 271 aa, synthetic construct
MHHHHHHHHENLYFQSGALGLPRREKNRVAARKCRAKFKNLLQHYREVAAAKSSENDRLRLLLKLENFQQRLQSLWTLARPFCPPLLATASQMQMVVLPCLGFTLLLWSQVSGAQGQEFHFGPCQVKGVVPQKLWEAFWAVKDTMQAQDNNTSCRLLQQEGLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 49 - protein, 270 aa, synthetic construct
MHHHHHHHHENLYFQSGALGLPRREKNRVAARKRAKFKNLLQHYREVAAAKSSENDRLRLLLKLENFQQRLQSLWTLARPFCPPLLATASQMQMVVLPCLGFTLLLWSQVSGAQGQEFHFGPCQVKGVVPQKLWEAFWAVKDTMQAQDNNTSCRLLQQEGLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 50 - protein, 274 aa, synthetic construct
MHHHHHHHHENLYFQSGALGLPKRYKNRVASRKCRAKFKQAETQKLISEIDLLRKQNEQLKHKLEQLLENFQQRLQSLWTLARPFCPPLLATASQMQMVVLPCLGFTLLLWSQVSGAQGQEFHFGPCQVKGVVPQKLWEAFWAVKDTMQAQDNNTSCRLLQQEGLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 51 - protein, 273 aa, synthetic construct
MHHHHHHHHENLYFQSGALGLPKRYKNRVASRKRAKFKQAETQKLISEIDLLRKQNEQLKHKLEQLLENFQQRLQSLWTLARPFCPPLLATASQMQMVVLPCLGFTLLLWSQVSGAQGQEFHFGPCQVKGVVPQKLWEAFWAVKDTMQAQDNNTSCRLLQQEGLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 52 - protein, 274 aa, synthetic construct
MHHHHHHHHENLYFQSGALGLPRREKNRVAARKCRAKFKNAETQKLISEIDLLRKQNEQLKHKLEQLLENFQQRLQSLWTLARPFCPPLLATASQMQMVVLPCLGFTLLLWSQVSGAQGQEFHFGPCQVKGVVPQKLWEAFWAVKDTMQAQDNNTSCRLLQQEGLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 53 - protein, 273 aa, synthetic construct
MHHHHHHHHENLYFQSGALGLPRREKNRVAARKRAKFKNAETQKLISEIDLLRKQNEQLKHKLEQLLENFQQRLQSLWTLARPFCPPLLATASQMQMVVLPCLGFTLLLWSQVSGAQGQEFHFGPCQVKGVVPQKLWEAFWAVKDTMQAQDNNTSCRLLQQEGLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 54 - protein, 223 aa, synthetic construct
MHHHHHHHHENLYFQSGALGLPKRYKNRVASRKCRAKFKQLLQHYREVAAAKSSENDRLRLLLKQLEGQEFHFGPCQVKGVVPQKLWEAFWAVKDTMQAQDNITSARLLQQEVLQNVSDAESCYLVHTLLEFYLKTVFKNHHNRTVEVRTLKSFSTLANNFVLIVSQLQPSQENEMFSIRDSAHRRFLLFRRAFKQLDVEAALTKALGEVDILLTWMQKFYKL

SEQ ID NO: 55 - protein, 222 aa, synthetic construct
MMHHHHHHHHENLYFQSGALGLPKRYKNRVASRKRAKFKQLLQHYREVAAAKSSENDRLRLLLKLEGQEFHFGPCQVKGVVPQKLWEAFWAVKDTMQAQDNITSARLLQQEVLQNVSDAESCYLVHTLLEFYLKTVFKNHHNRTVEVRTLKSFSTLANNFVLIVSQLQPSQENEMFSIRDSAHRRFLLFRRAFKQLDVEAALTKALGEVDILLTWMQKFYKL

SEQ ID NO: 56 - protein, 222 aa, synthetic construct
MHHHHHHHHENLYFQSGALGLPRREKNRVAARKCRAKFKNLLQHYREVAAAKSSENDRLRLLLKLEGQEFHFGPCQVKGVVPQKLWEAFWAVKDTMQAQDNITSARLLQQEVLQNVSDAESCYLVHTLLEFYLKTVFKNHHNRTVEVRTLKSFSTLANNFVLIVSQLQPSQENEMFSIRDSAHRRFLLFRRAFKQLDVEAALTKALGEVDILLTWMQKFYKL

SEQ ID NO: 57 - protein, 221 aa, synthetic construct
MHHHHHHHHENLYFQSGALGLPRREKNRVAARKRAKFKNLLQHYREVAAAKSSENDRLRLLLKLEGQEFHFGPCQVKGVVPQKLWEAFWAVKDTMQAQDNITSARLLQQEVLQNVSDAESCYLVHTLLEFYLKTVFKNHHNRTVEVRTLKSFSTLANNFVLIVSQLQPSQENEMFSIRDSAHRRFLLFRRAFKQLDVEAALTKALGEVDILLTWMQKFYKL

SEQ ID NO: 58 - protein, 225 aa, synthetic construct
MHHHHHHHHENLYFQSGALGLPKRYKNRVASRKCRAKFKQAETQKLISEIDLLRKQNEQLKHKLEQLLEGQEFHFGPCQVKGVVPQKLWEAFWAVKDTMQAQDNITSARLLQQEVLQNVSDAESCYLVHTLLEFYLKTVFKNHHNRTVEVRTLKSFSTLANNFVLIVSQLQPSQENEMFSIRDSAHRRFLLFRRAFKQLDVEAALTKALGEVDILLTWMQKFYKL

SEQ ID NO: 59 - protein, 224 aa, synthetic construct
MHHHHHHHHENLYFQSGALGLPKRYKNRVASRKRAKFKQAETQKLISEIDLLRKQNEQLKHKLEQLLEGQEFHFGPCQVKGVVPQKLWEAFWAVKDTMQAQDNITSARLLQQEVLQNVSDAESCYLVHTLLEFYLKTVFKNHHNRTVEVRTLKSFSTLANNFVLIVSQLQPSQENEMFSIRDSAHRRFLLFRRAFKQLDVEAALTKALGEVDILLTWMQKFYKL

SEQ ID NO: 60 - protein, 225 aa, synthetic construct
MHHHHHHHHENLYFQSGALGLPRREKNRVAARKCRAKFKNAETQKLISEIDLLRKQNEQLKHKLEQLLEGQEFHFGPCQVKGVVPQKLWEAFWAVKDTMQAQDNITSARLLQQEVLQNVSDAESCYLVHTLLEFYLKTVFKNHHNRTVEVRTLKSFSTLANNFVLIVSQLQPSQENEMFSIRDSAHRRFLLFRRAFKQLDVEAALTKALGEVDILLTWMQKFYKL

SEQ ID NO: 61 - protein, 224 aa, synthetic construct
MHHHHHHHHENLYFQSGALGLPRREKNRVAARKRAKFKNAETQKLISEIDLLRKQNEQLKHKLEQLLEGQEFHFGPCQVKGVVPQKLWEAFWAVKDTMQAQDNITSARLLQQEVLQNVSDAESCYLVHTLLEFYLKTVFKNHHNRTVEVRTLKSFSTLANNFVLIVSQLQPSQENEMFSIRDSAHRRFLLFRRAFKQLDVEAALTKALGEVDILLTWMQKFYKL